(19) United States
(12) Patent Application Publication
(10) Pub. No.: US 2008/0032985 A1
(43) Pub. Date: Feb. 7, 2008

(54) 4-Substituted Quinoline Derivatives, Method and Intermediates for Their Preparation and Pharmaceutical Compositions Containing Them

(76) Inventors: Michel Tabart, La Norville (FR); Fabrice Viviani, Louvres (FR); Serge Mignani, Chatenay Malabry (FR); Baptiste Ronan, Clamart (FR)

Correspondence Address: MARSH, FISCHMANN & BREYFOGLE LLP, 3151 SOUTH VAUGHN WAY, SUITE 411, AURORA, CO, 80014, US

(21) Appl. No.: 11/571,407

(22) PCT Filed: Jun. 24, 2005
(86) PCT No.: PCT/FR05/01598

§ 371 Date: Dec. 28, 2006

(30) Foreign Application Priority Data
Jun. 29, 2004 (FR) 0407124

(51) Int. Cl. A61K 31/47; A61K 31/4375; A61K 31/502; A61K 31/517; A61P 31/00; C07D 215/12; C07D 237/28; C07D 239/72; C07D 471/04

(52) U.S. Cl. 514/248; 514/266.1; 514/300; 514/311; 544/235; 544/283; 546/122; 546/152

(57) ABSTRACT

The present invention relates to 4-substituted quinoline derivatives of general formula I:

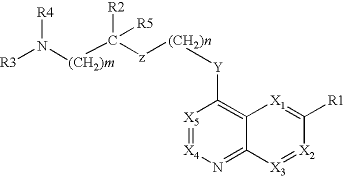

which are active as antimicrobials. The invention also relates to the method and intermediates for their preparation and the pharmaceutical compositions containing them.

The present invention relates to 4-substituted quinoline derivatives of general formula:

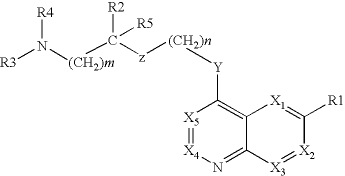

which are active as antimicrobials. The invention also relates to the method and intermediates for their preparation and the pharmaceutical compositions containing them.

In patent applications WO 99/37635 and WO 00/43383 have been described antimicrobial quinolylpropylpiperidine derivatives of general formula:

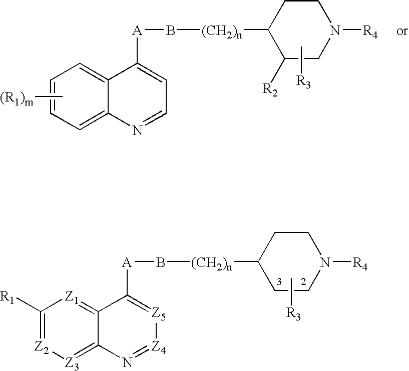

or
in which the radical R₁ is in particular (C1-6) alkoxy, R₂ is hydrogen, R₃ is at the 2 or 3 position and represents (C1-6) alkyl which may be optionally substituted with 1 to 3 substituents chosen from thiol, halogen, alkylthio, trifluoromethyl, carboxyl, alkyloxycarbonyl, alkylcarbonyl, alkenyloxycarbonyl, alkenylcarbonyl, hydroxyl optionally substituted with alkyl, R₄ is a group —CH₂—R₅ for which R₅ is selected from alkyl, hydroxyalkyl, alkenyl, alkynyl, tetrahydrofuryl, optionally substituted phenylalkyl, optionally substituted phenylalkenyl, optionally substituted heteroarylalkyl, optionally substituted heteroaroyl, n is 0 to 2, m is 1 or 2 and A and B are in particular oxygen, sulfur, sulfinyl, sulfonyl, NR₁₁, CR₆R₇ for which R₆ and R₇ represent H, thiol, alkylthio, halo, trifluoromethyl, alkenyl, alkenylcarbonyl, hydroxyl, amino, and Z₁ to Z₅ are N or CR₁ₐ.

Other applications, in particular WO 00/21952, WO 00/21948, WO 01/07432,

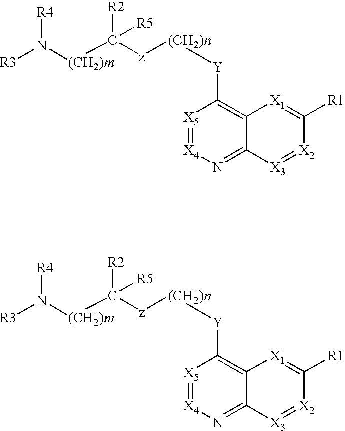

WO 01/07433, WO 01/25227, WO 03/010138, WO 02/40474 or WO 02/072572 describe other 4-(quinolylpropyl)-piperidine derivatives, substituted in particular at the 3 position or disubstituted at the 4 position, which are active in the same field. Moreover, mention may also be made of European application EP 30044 which describes related derivatives that are active in another field. All these applications describe compounds containing a chain attached to the 4 position of the quinoline and which contain a substituted nitrogen-containing heterocycle.

It has now been found, and that is what constitutes the subject of the present invention, that the compounds derived from 4-substituted quinoline of general formula (I), in which:
1) X₁, X₂, X₃, X₄ and X₅ represent >C-R′₁ to >C-R′₅ respectively, or alternatively at most one of them represents a nitrogen atom,

R₁, R′₁, R′₂, R′₃, R′₄ and R′₅ are identical or different and represent a hydrogen or halogen atom or an alkyl, cycloalkyl, phenyl, phenylthio, mono- or bicyclic heteroaryl or heteroarylthio, OH, SH, alkyloxy, difluoromethoxy, trifluoromethoxy, alkylthio, tri-fluoromethylthio, cycloalkyloxy, cycloalkylthio, acyl, acyloxy, acylthio, cyano, carboxyl, alkyloxycarbonyl, cycloalkyloxycarbonyl, nitro, —NRaRb or —CONRaRb radical (for which Ra and Rb can represent a hydrogen atom, an alkyl, cycloalkyl, phenyl, mono- or bicyclic heteroaryl radical or Ra and Rb form together with the nitrogen atom to which they are attached a 5- or 6-membered heterocycle which may optionally contain another heteroatom chosen from O, S or N and carrying, where appropriate, an alkyl, phenyl or mono- or bicyclic heteroaryl substituent on the nitrogen atom or, where appropriate, in which the sulfur atom is oxidized to the sulfinyl or sulfonyl state),

or represent a methylene radical substituted with fluoro, hydroxyl, alkyloxy, alkylthio, cycloalkyloxy, cycloalkylthio, phenyl, mono- or bicyclic heteroaryl, carboxyl, alkyloxycarbonyl, cycloalkyloxycarbonyl, —NRaRb or —CONRaRb for which Ra and Rb are as defined above,

or represent phenoxy, heterocyclyloxy, benzyloxy, heterocyclylmethyloxy,

or alternatively R₁ may also represent difluoromethoxy,

or a radical having the structure —C_{m′}F_{2m′+1}, —SC_{m′}F_{2m′+1} or —OC_{m′}F_{2m′+1} for which m′ is an integer from 1 to 6 or alternatively R′₅ may also represent trifluoroacetyl;

n is equal to 0, 1 or 2;

m is equal to 0, 1 or 2;

Y represents a group CHR, C=O, CROH, CRNH₂, CRF or CF₂,

R being a hydrogen atom or a (C₁₋₆) alkyl radical;

Z represents a group CH₂ or alternatively Z represents an oxygen atom, a sulfur atom or a group NH when n and m are equal to 1 or 2 and when Y represents a group CROH, CRNH₂, CRF or CF₂;

R₂ represents a radical —CO₂R, —CH₂CO₂R, —CH₂—CH₂OH, CH₂OH, CH₂—CH₂CO₂R, —CONH₂, —CH₂—CONH₂, —CH₂—CH₂—CONH₂, —CH₂—NH₂, —CH₂—CH₂—NH₂ or —CH₂—CH₂—CH₂—NH₂, R being as defined above;

R₃ represents a radical phenyl, heteroaryl, alk-R^{o}₃ for which alk is an alkylene radical and

R^{o}₃ represents hydrogen, halogen, hydroxyl, alkyloxy, alkylthio, alkylsulfinyl, alkylsulfonyl, alkylamino, dialkylamino, cycloalkyl, cycloalkyloxy, cyclo-alkylthio, cycloalkylsulfinyl, cycloalkylsulfonyl, cycloalkylamino, N-cycloalkyl-N-alkylamino, —N-(cycloalkyl)₂, acyl, cycloalkylcarbonyl, phenyl, phenoxy, phenylthio, phenylsulfinyl, phenylsulfonyl, phenylamino, N-alkyl-N-phenylamino, N-cycloalkyl-N-phenylamino, —N-(phenyl)2, phenylalkyloxy, phenylalkyl-thio, phenylalkylsulfinyl, phenylalkylsulfonyl, phenyl-alkylamino, N-alkyl-N-phenylaminoalkyl, N-cycloalkyl-N-phenylalkylamino, benzoyl, heteroaryl, heteroaryl-oxy, heteroarylthio, heteroarylsulfinyl, heteroaryl-sulfonyl, heteroarylamino, N-alkyl-N-heteroarylamino, N-cycloalkyl-N-heteroarylamino, heteroarylcarbonyl, heteroarylalkyloxy, heteroarylalkylthio, heteroaryl-alkylsulfinyl, heteroarylalkylsulfonyl, heteroaryl-alkylamino, N-alkyl-N-heteroarylaminoalkyl, N-cyclo-alkyl-N-heteroarylaminoalkyl (the heteroaryl parts mentioned above being mono- or bicyclic), carboxyl, alkyloxycarbonyl, —NRaRb or —CO—NRaRb for which Ra and Rb respectively represent hydrogen, alkyl, cycloalkyl, phenyl, mono- or bicyclic heteroaryl, or one of Ra or Rb represents hydroxyl, alkyloxy, cycloalkyloxy, or Ra and Rb form together with the nitrogen atom to which they are attached a 5- or 6-membered heterocycle which may optionally contain another heteroatom chosen from O, S and N and carrying, where appropriate, an alkyl, phenyl or mono- or bicyclic heteroaryl substituent on the nitrogen atom or where appropriate in which the sulfur atom is oxidized to the sulfinyl or sulfonyl state,

or alternatively R^{o}₃ represents —CR′b=CR′c-R′a for which R′a represents phenyl, phenylalkyl, heteroaryl, heteroarylalkyl, phenoxyalkyl, phenylthioalkyl, phenyl-sulfinylalkyl, phenylsulfonylalkyl, phenylaminoalkyl, N-alkyl-N-phenylaminoalkyl, heteroaryloxyalkyl, hetero-arylthioalkyl, heteroarylsulfinylalkyl, heteroaryl-sulfonylalkyl, heteroarylaminoalkyl, N-alkyl-N-hetero-arylaminoalkyl, heteroarylthio, heteroarylsulfinyl, heteroarylsulfonyl, (the heteroaryl parts mentioned above being mono- or bicyclic), phenylthio, phenyl-sulfinyl, phenylsulfonyl, and for which R′b and R′c represent hydrogen, alkyl or cycloalkyl,

or alternatively R₃ represents a radical —C=C-Rd for which Rd is alkyl, phenyl, phenylalkyl, phenoxyalkyl, phenylthioalkyl, N-alkyl-N-phenylaminoalkyl, hetero-aryl, heteroarylalkyl, heteroaryloxyalkyl, hetero-arylthioalkyl, heteroarylaminoalkyl, N-alkyl-N-heteroarylaminoalkyl, (the heteroaryl parts mentioned above being mono- or bicyclic),

or alternatively R^{o}₃ represents a radical —CF₂-phenyl or mono- or bicyclic —CF₂-heteroaryl,

it being understood that the phenyl, benzyl, benzoyl or heteroaryl radicals or portions mentioned above are optionally substituted on the ring with 1 to 4 substituents chosen from halogen, hydroxyl, alkyl, alkyloxy, alkyloxyalkyl, haloalkyl, trifluoromethyl, trifluoromethoxy, trifluoromethylthio, carboxyl, alkyl-oxycarbonyl, cyano, alkylamino, —NRaRb for which Ra and Rb are as defined above, phenyl, hydroxyalkyl, alkylthioalkyl, alkylsulfinylalkyl, alkylsulfonylalkyl;

R₄ represents a hydrogen atom or an alkyl radical optionally substituted with R₆, where R₆ represents an OH, NH₂ or COOH radical, or a fluorine atom; and

R₅ is a hydrogen atom or an alkyl group;

it being understood that the alkyl or acyl radicals and portions contain (unless specifically stated) 1 to 10 carbon atoms in the form of a straight or branched chain and that the cycloalkyl radicals contain 3 to 6 carbon atoms;

in their enantiomeric or diastereoisomeric forms or mixtures of these forms, and/or where appropriate in E or Z form or mixtures thereof, and their salts, are very potent antibacterial agents.
2) Among the compounds of general formula (I), there are preferred those in which:

R₁, R′₁, R′₂, R′₃, R′₄ and R′₅ are identical or different and represent a hydrogen or halogen atom or an alkyl or alkyloxy radical, or represent a methylene radical substituted with alkyloxy:

m and n are equal to 1 or 2; and

R₃ represents a radical alk-R^{o}₃ for which alk is an alkylene radical and R^{o}₃ represents alkyloxy, alkylthio, alkylamino, dialkylamino, cycloalkyloxy, cycloalkylthio, cycloalkylamino, N-cycloalkyl-N-alkylamino, —N-(cycloalkyl) 2, phenyl, phenoxy, phenylthio, phenylamino, N-alkyl-N-phenylamino, N-cycloalkyl-N-phenylamino, phenylalkyloxy, phenyl-alkylthio, phenylalkylamino, N-alkyl-N-phenyl-aminoalkyl, N-cycloalkyl-N-phenylalkylamino, hetero-aryloxy, heteroarylthio, heteroarylamino, N-alkyl-N-heteroarylamino, N-cycloalkyl-N-heteroarylamino, heteroarylcarbonyl, heteroarylalkyloxy, heteroaryl-alkylthio, heteroarylalkylamino, N-alkyl-N-heteroarylaminoalkyl, N-cycloalkyl-N-heteroarylamino-alkyl, (the heteroaryl parts cited above being mono- or bicyclic), —NRaRb or —CO—NRaRb for which Ra and Rb are defined as in point 1,

or alternatively R^{o}₃ represents —CR′b=CR′c-R′a for which R′a represents phenyl, phenylalkyl, heteroaryl or heteroarylalkyl, phenoxyalkyl, phenylthioalkyl, phenyl-aminoalkyl, N-alkyl-N-phenylaminoalkyl, heteroaryloxy-alkyl, heteroarylthioalkyl, heteroarylaminoalkyl, N-alkyl-N-heteroarylaminoalkyl, heteroarylthio, (the heteroaryl parts cited above being mono- or bicyclic), or phenylthio, and for which R′b and R′c represent hydrogen, alkyl or cycloalkyl,

or alternatively R^{o}₃ represents a radical —C—C—Rd for which Rd is alkyl, phenyl, phenylalkyl, phenoxyalkyl, phenylthioalkyl, N-alkyl-N-phenylaminoalkyl, heteroaryl, heteroarylalkyl, heteroaryloxyalkyl, heteroaryl-thioalkyl, heteroarylaminoalkyl, N-alkyl-N-heteroaryl-aminoalkyl, (the heteroaryl parts cited above being mono- or bicyclic),

or alternatively R^{o}₃ represents a radical —CF₂-phenyl or mono- or bicyclic —CF₂-heteroaryl;

it being understood that the phenyl, benzyl, benzoyl or heteroaryl radicals or portions mentioned above may be optionally substituted as envisaged in point 1;

R₂, R₄, R₅, Y and Z are as defined in point 1;

in their enantiomeric or diastereoisomeric forms or mixtures of these forms, and/or where appropriate in E or Z form or mixtures thereof, and their salts.
3) Among the compounds of general formula (I), there are also preferred those in which:

R₁, R′₁, R′₂, R′₃, R′₄ and R′₅ are identical or different and represent a hydrogen or halogen atom or an alkyl or alkyloxy radical, or represent a methylene radical substituted with alkyloxy;

m and n are equal to 1;

Y represents a group CH₂, CHOH, CHF, CHNH₂ or C=O;

R₂ represents a radical COOR, CH₂—COOR, CH₂OH or CH₂CH₂OH, R being as defined in point 1;

Z represents a group CH₂;

R₃ represents a radical alk-R^{o}₃ for which alk is an alkylene radical and R^{o}₃ represents cycloalkyloxy, cycloalkylthio, phenyl, phenoxy, phenylthio, phenylalkyloxy, phenylalkylthio, heteroaryloxy, heteroarylthio, heteroarylalkyloxy, heteroarylalkyl-thio, (the heteroaryl parts cited above being mono- or bicyclic),

or alternatively R^{o}₃ represents —CR′b=CR′c-R′a for which R′a represents phenyl, phenylthioalkyl, hetero-aryl, heteroarylalkyl, phenoxyalkyl, phenylthioalkyl, heteroaryloxyalkyl, heteroarylthioalkyl (the heteroaryl parts cited above being mono- or bicyclic), or phenylthio, and for which R′b and R+c represent hydrogen, alkyl or cycloalkyl,

or alternatively R^{o}₃ represents a radical —C—C—Rd for which Rd is alkyl, phenyl, phenylalkyl, phenoxyalkyl, phenylthioalkyl, N-alkyl-N-phenylaminoalkyl, mono- or bicyclic heteroaryl, heteroarylalkyl, heteroaryloxy-alkyl, heteroarylthioalkyl, (the heteroaryl parts cited above being mono- or bicyclic);

R₄ represents a hydrogen atom or an alkyl radical optionally substituted with R₆, where R₆ represents an OH radical or a fluorine atom;

R₅ is a hydrogen atom or an alkyl group; it being understood that the phenyl, benzyl, benzoyl or heteroaryl radicals or portions mentioned above may be optionally substituted as envisaged above;

in their enantiomeric or diastereoisomeric forms or mixtures of these forms, and/or where appropriate in Z or E form or mixtures thereof, and their salts.
4) Among the compounds of general formula (I), there may be mentioned in particular those in which:

R₁, R′₁ R′₂, R′₃, R′₄ and R′₅ are identical or different and represent a hydrogen or halogen atom or an alkyl or alkyloxy radical or represent a methylene radical substituted with alkyloxy;

m and n are equal to 1;

Y and Z represent a group CH₂;

R₂ represents a radical COOR or CH₂—COOR, R being as defined in point 1;

R₃ and R₄ are as defined above in point 3;

R₅ is a hydrogen atom;

in their enantiomeric or diastereoisomeric forms or mixtures of these forms, and/or where appropriate in Z or E form or mixtures thereof, and their salts.
5) Among the compounds of general formula (I), the subject of the invention is most particularly any one of those whose names follow:

ethyl (RS)-2-{[(E)-3-(2,5-difluorophenyl)allyl-amino]methyl}-5-(3-fluoro-6-methoxyquinolin-4-yl)-pentanoate;

ethyl (RS)-2-{[(E)-3-(2,5-difluorophenyl)allyl-amino]methyl}-5-(3-fluoro-6-methoxyquinolin-4-yl)-pentanoate;

(RS)-2-{[(E)-3-(2,5-difluorophenyl)allylamino]-methyl}-5-(3-fluoro-6-methoxyquinolin-4-yl)pentanoic acid;

2-{[(E)-3-(2,5-difluorophenyl)allylamino]methyl}-5-(3-fluoro-6-methoxyquinolin-4-yl)pentanoic acid;

2-{[(E)-3-(2,5-difluorophenyl)allylamino]methyl-}5-(3-fluoro-6-methoxyquinolin-4-yl)pentanoic acid;

(RS)-2-{[3-(2,5-difluorophenyl)propylamino]-methyl}-5-(3-fluoro-6-methoxyquinolin-4-yl)pentanoic acid;

ethyl (RS)-2-({N-[(E)-3-(2,5-difluorophenyl)allyl]-N-methylamino}methyl)-5-(3-fluoro-6-methoxyquinolin-4-yl)pentanoate;

sodium (RS)-2-({N-[(E)-3-(2,5-difluorophenyl)-allyl]-N-methylamino}methyl)-5-(3-fluoro-6-methoxy-quinolin-4-yl)pentanoate;

(RS)-5-(3-fluoro-6-methoxyquinolin-4-yl)-2-{[2-(thiophen-2-ylsulfanyl)ethylamino]methyl}pentanoic acid;

(RS)-2-{[2-(2,5-difluorophenylsulfanyl)ethylamino]-methyl}-5-(3-fluoro-6-methoxyquinolin-4-yl)pentanoic acid;

(RS)-2-{[2-(2,5-difluorophenoxy)ethylamino]methyl}-5-(3-fluoro-6-methoxyquinolin-4-yl)pentanoic acid;

(RS)-2-{[N-[(E)-3-(2,5-difluorophenyl)allyl]-N-(2-fluoroethyl)amino]methyl}-5-(3-fluoro-6-methoxy-quinolin-4-yl)pentanoic acid;

(RS)-2-{[N-[(E)-3-(2,5-difluorophenyl)allyl]-N-(2-hydroxyethyl)amino]methyl}-5-(3-fluoro-6-methoxy-quinolin-4-yl)pentanoic acid;

in their enantiomeric or diastereoisomeric forms or mixtures of these forms, and/or where appropriate in Z or E form or mixtures thereof, and their satls.

According to the invention, the products of general formula (I) may be obtained according to the method in which the chain R₃ defined above is condensed with the 4-substituted quinoline derivative of general formula (II)

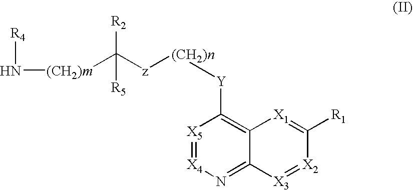

in which X₁, X₂, X₃, X₄, X₅, R₁, R₂, Y, Z, m, n, R₄ and R₅ are as defined above, R₂ being protected when it carries a carboxyl radical, and then where appropriate the group protecting the carboxyl radical is removed, optionally the enantiomeric and diastereoisomeric forms and/or where appropriate the Z or E forms are separated and optionally the product obtained is converted to a salt.

Preferably, the condensation of the chain R₃ with the nitrogen is carried out by the action of a derivative of general formula (IIa):

R₃-X (IIa)

in which R₃ is defined as above and X represents a halogen atom, a methylsulfonyl radical, a trifluoromethylsulfonyl radical or a p-toluenesulfonyl radical.

Preferably, when R₃ represents a radical -alk-R^{o}₃ for which alk is an alkyl radical and R^{o}₃ represents a radical —C—C—Rd in which Rd is as defined above, a condensation of an alkynyl halide HC≡C-alk-X for which alk is defined as above and X is a halogen atom is carried out, followed by substitution of the chain with an appropriate radical Rd.

Preferably still, when R₃ represents a radical -alk-R^{o}₃ for which alk is an alkyl radical and R^{o}₃ represents a phenoxy, phenylthio, phenylamino, heteroaryloxy, heteroarylthio or heteroarylamino radical, the reaction is carried out by constructing the chain by first condensing a chain HO-alk-X for which X is a halogen atom, and then either by converting the hydroxyalkyl chain obtained to a haloalkyl, methanesulfonylalkyl or p-toluenesulfonylalkyl chain and finally by causing an aromatic derivative having the structure R₃H or R₃H₂ to act in a basic medium, or by causing the aromatic derivative to act directly under dehydration conditions.

According to one embodiment, for the preparation of the compounds of general formula (I) in which R₄ represents an alkyl group optionally substituted with R₆, a product of general formula (I) where R₄ represents a hydrogen atom is subjected to the action of an appropriate alkylating reagent.

According to the invention, the derivatives of general formula (II) for which Y is a group CHR, Z is a group CH₂ and m and n are defined as above, are prepared by condensing a heteroaromatic derivative of general formula (III):

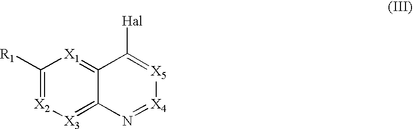

in which R₁, X₁, X₂, X₃, X₄ and X₅ are defined as above and Hal represents a halogen atom, with a derivative of general formula (IV):

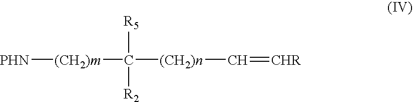

in which p is a group protecting the amino functional group and R, m, n, R₅ and R₂ are defined as above or R₂ represents a protected radical if R₂ represents or carries a carboxylic acid functional group, followed by the removal of the protecting groups and/or followed by the conversion, by a subsequent operation, of the substituents of the aromatic bicycle of general formula (II) thus obtained, to give the expected derivative carrying the radical R₁, R′₁, R′₂, R′₃, R′₄, R′₅, and where appropriate removing the protecting radical(s) still present in the molecule.

The subject of the invention is also the derivatives of general formula (II) and (IV) as defined above.

The subject of the invention is also, as medicaments, the derivatives of general formula (I) as defined above.

The subject of the invention is also a pharmaceutical composition which contains at least one medicament of general formula (I) in the pure state or in combination with one or more compatible and pharmaceutically acceptable diluents and/or adjuvants.

According to the invention, the compounds of general formula (I) may be prepared by condensing, with a compound of general formula (II)

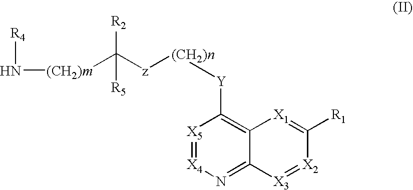

in which R₁, X₁, X₂, X₃, X₄, X₅, Y, n, Z, R₅, R₂, m and R₄ are defined as above,

the chain R₃, R₂ being protected when it carries a carboxyl radical, followed where appropriate by the removal of the group protecting the carboxyl, optionally the separation of the enantiomeric or diastereoisomeric forms and/or where appropriate of the syn or anti forms and optionally the conversion of the product obtained to a salt.

The condensation of the chain R₃ with the nitrogen atom is advantageously carried out by the action of a derivative of general formula:

R₃-X (IIa)

in which R₃ is as defined above and X represents a halogen atom, a methylsulfonyl radical, a trifluoromethylsulfonyl or p-toluenesulfonyl radical, the procedure being carried out in an anhydrous, preferably inert, medium, in an organic solvent such as an amide (dimethylformamide for example), a ketone (acetone for example) or a nitrile (acetonitrile for example) in the presence of a base such as a nitrogenous organic base (for example triethylamine) or an inorganic base (for example an alkali metal carbonate such as potassium carbonate) at a temperature of between 20° C. and the reflux temperature of the solvent.

The amino functional group is optionally protected according to the customary methods compatible with the remainder of the molecule or the reaction; the protection is performed for example with a protecting radical chosen from benzyl, t-butoxycarbonyl and benzyloxycarbonyl groups, and this functional group is released prior to the condensation with the derivative of formula (IIa), in particular by acid hydrolysis.

Preferably, a derivative of general formula (IIa) for which X is a chlorine, bromine or iodine atom is caused to act.

General conditions under which it is possible to carry out the condensation between the derivatives of general formulae (II) and (IIa) may also be found in application WO 02/40474.

When R₃ is a radical -alk-R′₃ in which R′₃ is a group —C≡C—Rd, in which Rd is as defined above, an alkynyl halide is intermediately condensed and then the desired radical is condensed with the alkyne thus obtained.

When R₃ represents a radical -alk-R^{o}₃ for which alk is an alkyl radical and R^{o}₃ represents a phenoxy, phenylthio, phenylamino, heteroaryloxy, heteroarylthio or heteroarylamino radical, it is also possible to construct the chain by first condensing a chain HO-alk-X for which X is a halogen atom, preferably iodine, under the conditions described above for the reaction of the product of general formula (IIa), and then, where appropriate, by converting the hydroxyalkyl chain to a haloalkyl, methanesulfonylalkyl or p-toluene-sulfonylalkyl chain and finally by causing an aromatic derivative having the structure R^{o}₃H or R^{o}₃H₂ to act in a basic medium or by causing the aromatic derivative to act directly under dehydration conditions.

The conversion of the hydroxylated chain to a haloalkyl or p-toluenesulfonyl chain is carried out according to the customary halogenation or sulfonylation methods, in particular a halogenating agent such as thionyl chloride, the halogenated derivatives of phosphorus (phosphorus trichloride or tribromide for example) or a sulfonylating agent such as for example methanesulfonyl chloride, p-toluenesulfonyl chloride or trifluoro-methanesulfonic anhydride is caused to act. The reaction is carried out in an organic solvent such as a chlorinated solvent (dichloromethane or chloroform for example), at a temperature of between 0 and 60° C. In some cases, it may be advantageous to carry out the procedure in the presence of a base such as pyridine or triethylamine.

The reaction of the aromatic derivative R^{o}₃H or R^{o}₃H₂ is advantageously carried out as described above for the action of the derivative of general formula (IIa), in an organic solvent such as an amide (dimethylformamide for example), a ketone (acetone for example), a nitrile (acetonitrile for example), in the presence of a base such as a nitrogenous organic base (for example triethylamine) or an inorganic base (alkali metal carbonate: potassium carbonate for example) at a temperature of between 20° C. and the reflux temperature of the reaction mixture. It may be advantageous to carry out the procedure in the presence of potassium iodide. It is also possible to carry out the procedure in an ether (tetrahydrofuran for example) under conditions using, for example, diethyl azodicarboxylate and triphenylphosphine.

It is understood that, if the radicals R₃ carry carboxyl or amino substituents, the latter are protected beforehand, and then released after the reaction. The procedure is carried out according to methods well known to a person skilled in the art which do not adversely affect the remainder of the molecule, in particular according to the methods described by T. W. Greene and P. G. M. Wuts, Protective Groups in Organic Synthesis (2nd ed.), A. Wiley—Interscience Publication (1991), or by Mc Omie, Protective Groups in Organic Chemistry, Plenum Press (1973).

The protected carboxyl radical carried where appropriate by R₂ may be chosen from easily hydrolyzable esters. By way of example, there may be mentioned methyl, benzyl or tert-butyl esters, or alternatively phenylpropyl or allyl esters. Optionally, the protection of the carboxyl radical is carried out simultaneously with the reaction.

The introduction and the removal of these protecting radicals are carried out according to methods known to a person skilled in the art.

According to the invention, the condensation of the chain R₃ with the nitrogen atom may also be carried out by the action of a derivative which is a precursor of R₃ containing at the chain end an aldehyde functional group, the carbon atom thereof forming an integral part of R₃. The procedure is carried out in an anhydrous medium in an inert solvent such as an ether, for example diethyl ether, or a halogenated solvent, for example dichloromethane, under reductive amination conditions, in the presence of a base as described above, and of a reducing agent such as a borohydride, for example sodium borohydride or sodium triacetoxy-borohydride.

According to the invention, the products of general formula (I) in which R₃ represents a radical alk-R^{o}₃ may also be prepared by a method according to which a reducing agent is reacted with a compound of general formula (I) in which R^{o}₃ represents a radical —CR′b=CR′c-R′a. The procedure is carried out by the action of hydrogen in the presence of an appropriate catalyst, in particular palladium. An example is presented later in the experimental section.

According to the invention, the products of general formula (I) in which R₄ represents an alkyl group optionally substituted with R₆ may be prepared by the action, on a product of general formula (I) in which R₄ represents a hydrogen atom, of an appropriate alkylating reagent.

The alkylating reagent may be in particular a halide or more generally a product of formula C—CH₂-R₆ in which X and R₆ are as defined above, which is caused to react in the presence of a base, for example an alkali metal carbonate, or alternatively an appropriate aldehyde which is caused to react under reductive amination conditions such as those described above.

According to the invention, the derivatives of general formula (I) for which R₂ is hydroxymethyl or hydroxyethyl may be prepared by the action of an appropriate reducing agent on a derivative for which R₂ is carboxyl or carboxymethyl or protected carboxyl or protected carboxymethyl. A ketone functional group which may be present should then be intermediately protected.

Also according to the invention, the products of general formula (I) for which R₂ is carboxymethyl or carboxyethyl may also be prepared from the derivatives for which R₂ is hydroxymethyl or hydroxyethyl, by the action on the latter of a halogenating or tosylating agent, and then of a cyanating agent and finally of an agent for hydrolyzing the nitrile.

Also according to the invention, the products of general formula (I) for which R₂ is —CH₂—NH₂, —(CH₂)₂—NH₂ or —(CH₂)₃—NH₂ may be prepared from the corresponding amides by reduction under conditions known to persons skilled in the art.

It is possible to carry out the reduction of the protected carboxyl according to the customary methods which do not adversely affect the remainder of the molecule, in particular by the action of a hydride (lithium aluminum hydride or diisobutyl aluminum hydride for example) in a solvent such as an ether (tetrahydrofuran for example) at a temperature of between 20 and 60°C. A ketone functional group which may be present is intermediately protected and then deprotected according to conventional methods known to a person skilled in the art, in particular via a cyclic or noncyclic acetal.

The reduction of the free carboxyl may be carried out according to methods which are also known to a person skilled in the art, for example by hydrogenation in the presence of a rhodium- or ruthenium-based catalyst, by the action of sodium borohydride in the presence of a Lewis acid or of lithium aluminum hydride in ether. Preferably, a ketone functional group which may be present is in this case also protected in an intermediate phase.

The conversion of the hydroxymethyl or hydroxyethyl radical to a carboxymethyl or carboxyethyl radical is carried out according to the customary methods which do not adversely affect the remainder of the molecule, in particular by the action of a halogenating agent such as for example thionyl chloride or phosphorus trichloride or phosphorus tribromide, or of a tosylating agent, followed by an alkali metal cyanide, for example potassium cyanide or sodium cyanide, in order to prepare the corresponding cyanomethyl derivative, followed by hydrolysis of the nitrile.

The halogenation may be carried out in a chlorinated solvent (dichloromethane or chloroform for example), at a temperature of between 0° C. and the reflux temperature of the solvent.

The amidation reaction with ammonia is carried out under the customary conditions known to persons skilled in the art. The procedure is preferably carried out starting with the acid, for example in the presence of dicyclohexylcarbodiimide and dimethylaminopyridine or hydroxybenzotriazole, in an ether, for example tetrahydrofuran, a chlorinated solvent, for example dichloromethane or dimethylformamide.

The reduction to an amine is likewise carried out under conventional conditions, for example by the action of a hydride such as lithium aluminum hydride, in an ether, for example tetrahydrofuran, or by the action of a borane in the presence of dimethyl sulfide.

The condensation of the chain R₃ with the nitrogen at the end of the chain does not require in principle that the nitrogen inside the chain is protected. Where appropriate, in the exceptional cases where this may prove necessary, a conventional group protecting the amine functional groups, such as those described in the book by T. W. Greene and P. G. M. Wuts cited above, may be used.

According to the invention, the products of general formula (I) in which R₅ represents an alkyl radical may be prepared by the action of an alkylating reagent in the presence of a base, on a product of general formula (I) in which R₅ represents a hydrogen atom and R₂ preferably represents a COOalkyl radical.

The alkylating reagent may be in particular an alkyl iodide and the base is a strong base and may be in particular an alkali metal amide such as lithium diisopropylamide. However, when the molecule contains an alkylable position other than that involved in the preceding reaction, in particular a secondary or primary nitrogen, an alcohol or a carbon carrying a carboxylic acid functional group, it will be necessary to protect it in an appropriate manner.

If the molecule contains an alkylable position which can directly enter into competition with that which it is desired to alkylate, the reaction will not be possible and the alkylation will be carried out at an earlier stage of the synthesis, as is described later.

According to the invention, the preparation of the products of general formula (II) for which Y is a groups CHR, Z is a group CH₂ and m and n are defined as above, is carried out by condensing a heteroaromatic compound of general formula

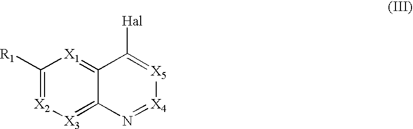

in which Hal represents a chlorine, bromine or iodine atom and R₁, X₁, X₂, X₃, X₄ and X₅ are defined as above, with a compound of general formula IV

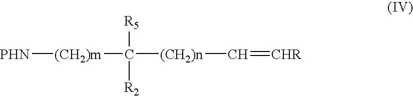

in which m, R₂, R₅, n and R are defined as above or R₂ represents a protected corresponding radical if R₂ represents or carries a carboxylic acid functional group, and P represents a group protecting the amino functional group, followed by the optional removal of the protecting groups and/or followed by the conversion, by a subsequent operation, of the substituents of the aromatic bicycle of general formula (II) thus obtained, to give the derivative carrying the expected radical R₁, R′₁, R′₂, R′₃, R′₄ and R′₅ and, where appropriate, removal the protecting radical(s) still present in the molecule.

P may be any group protecting the nitrogen atom, which is compatible with the reaction (t-butyloxycarbonyl, benzyloxycarbonyl for example). The groups protecting the acid functional groups are chosen from the customary groups whose introduction and removal do not affect the remainder of the molecule, in particular those mentioned in the references cited above.

The reaction may be carried in particular by the successive action on the derivative of general formula (IV) of an organoborane (9-borabicyclo[3.3.1]nonane for example) in a solvent such as an ether (tetrahydrofuran, dioxane for example) at a temperature of between −20 and 20° C. and then of the bicyclic derivative of general formula (III) for which Hal represents a chlorine atom or preferably a bromine or iodine atom, by analogy with methods described by Suzuki et al. Pure and Appl. Chem., 57, 1749 (1985). The reaction is generally carried out in the presence of a palladium salt ([bis(diphenylphosphino)ferrocene]-palladium chloride for example) and of a base such as potassium phosphate at a temperature of between 20° C. and the reflux temperature of the solvent.

According to the invention, the products of general formula (II) for which Y represents a group CHOH may be prepared by oxidation, in a basic medium, of the corresponding derivative for which Y is a group CHR. The oxidation is carried out by the action of oxygen, preferably in an inert solvent such as dimethyl sulfoxide, in the presence of tert-butanol and of a base such as potassium or sodium tert-butoxide at a temperature of between 0 and 100° C.

The derivatives of general formula (II) for which Y is a group CFR or CF₂ may be prepared by fluorination starting respectively with the derivative for which Y is a group CROH and that for which Y is a carbonyl group. The reaction is carried out in the presence of a sulfur fluoride [for example in the presence of an aminosulfur trifluoride (diethylaminosulfur trifluoride (Tetrahedron, 44, 2875 (1988)), bis(2-methoxyethyl)-aminosulfur trifluoride (Deoxofluor®), morpholinosulfur trifluoride for example) or alternatively in the presence of sulfur tetrafluoride (J. Org. Chem., 40, 3808 (1975)]. The fluorination reaction may also be carried out by means of a fluorinating agent such as hexafluoropropyldiethylamine (JP 2 039 546) or N-(2-chloro-1,1,2-trifluoroethyl)diethylamine.

The procedure is carried out in an organic solvent such as a chlorinated solvent (for example dichloromethane, dichloroethane, chloroform) or in an ether (tetrahydrofuran, dioxane for example) at a temperature of between −78 and 40° C. (preferably between 0 and 30° C.). It is advantageous to carry out the procedure in an inert medium (argon or nitrogen in particular).

The derivatives of general formula (II) for which Y is a carbonyl group may be prepared by oxidation of the corresponding derivative of general formula (II) for which is Y is a group CHOH. This oxidation is carried out for example by means of potassium permanganate, optionally in a sodium hydroxide solution (for example 3N sodium hydroxide), at a temperature of between −20 and 20° C., or alternatively by the action of oxalyl chloride in the presence of dimethyl sulfoxide, followed by the addition of an amine such as triethylamine, in an inert solvent such as dichloromethane or dimethyl sulfoxide at a temperature of between −60 and 20° C. by analogy with the method described by D. SWERN et al., J. Org. Chem. 44, 4148 (1979).

The derivative of general formula (II) for which Y is a group CRNH₂ may be prepared from the corresponding derivative CHOH which is converted to its tosylated derivative, with which ammonia is reacted. The procedure is carried out in an inert solvent such as N,N-dimethylformamide or dimethyl sulfoxide and preferably under pressure (2 to 20 atmospheres) at a temperature of between 20 and 100° C.

The tosyloxy derivative is obtained from the product of general formula (II) for which Y is CROH, by the action of tosyl chloride in pyridine, at a temperature of between −10 and 20° C.

The derivatives of formula (III) in which R₁, X₁, X₂, X₃, X₄ and X₅ are defined as above can be prepared by the methods described in application WO 02/40474.

The compounds of general formula (IV) may be prepared by the action of a compound of general formula (V)

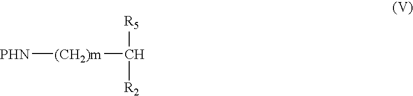

in which P, m, R₂ and R₅ are defined as above, R₂ preferably represents a COalkyl or COOp radical, p being a protecting group, on a compound of general formula (IV)

Hal-(CH₂)N—CH=CHR (VI)

in which n and R are defined as above and Hal represents a halogen atom, preferably a bromine atom.

The procedure is preferably carried out in the presence of a strong base, in particular an alkali metal amide, for example lithium bis(trimethylsilyl)amide, or a lithium compound, for example butyllithium, in an organic solvent which may be in particular an ether such as tetrahydrofuran or dioxane.

In the case where n=1, it may also be possible to carry out the procedure by condensing a derivative of general formula (V) as defined above with a product of the dibromoethane type of general formula:

BrCH₂—CHRBr (VI′)

in which R is defined as above, and then the product obtained is freed of hydrobromide by a method known to persons skilled in the art. Reference may be made for example to the method described by R. A. Bunce et al., Organic Preparations Procedure Internationale (1999-31 (1) p.99-106.

The compounds of general formula (V) in which R₅ represents an alkyl radical may be prepared by the action of an alkylating reagent in the presence of a base on the corresponding compounds in which R₅ is a hydrogen atom, under conditions identical to those described above for the preparation of the compounds of general formula (I).

According to the invention, the alkylation reaction may also be carried out on the compound of general formula (IV), that is to say by alkylation of a compound of formula IV in which R₅ represents a hydrogen atom, under the same conditions as above.

According to the invention, the compound of general formula (II) for which Z is an oxygen atom may be prepared starting with the compound of formula (VII)

H₂N—(CH₂)ₘCHOH—COOH (VII)

in which m is defined as above, whose amino functional group is protected in order to obtain a compound of general formula (VIII)

PHN—(CH₂)ₘ—CHOH—COOH (VIII)

in which P and m are defined as above, whose acid functional group is protected in order to obtain a compound of general formula (IX)

PHN—(CH₂)ₘ—CHOH—COOp (IX)

in which P and m are defined as above and p represents a protecting group, which is reacted with a compound of formula (X)

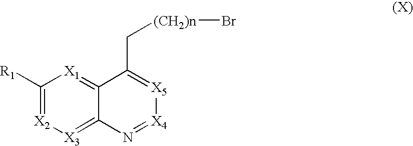

in which R₁, X₁, X₂, X₃, X₄ and X₅ are defined as above, in order to obtain a compound of general formula (IIp)

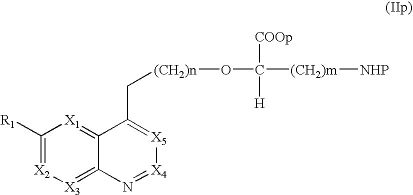

in which P, m, p, n, R₁, X₁, X₂, X₃, X₄ and X₅ are defined as above, which, where appropriate, is deprotected at the level of the amino functional group. The protecting groups and the methods for introducing them and where appropriate for removing them are those mentioned above.

The compound of formula (X) may be prepared by condensing the derivative lithiated at the 4-position of the heteroatomatic compound of general formula (III′)

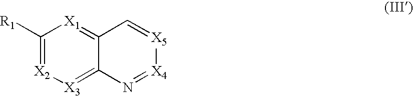

in which R₁, X₁, X₂, X₃, X₄ and X₅ are defined as above, with a compound of general formula (XI)

I—CH₂—(CH₂)—Br (XI)

in which n is defined as above.

The formation of the derivative lithiated at the 4-position of the compound (III′) occurs with the aid of a strong lithium-containing base such as butyllithium, sec-butyllithium, or preferably lithium diisopropylamide, in a solvent such as an ether, tetrahydrofuran for example, at a temperature of between −78° and −40°. The condensation of this lithium-containing derivative with the compound of formula (XI) is carried out in the same solvent, at a temperature of between −78° and 0° C.

The derivative of formula (III′) may be prepared according to a method described in patent application WO 02/40474.

The reaction of the compound of formula (X) with the compound of formula (IX) may be carried out in the presence of a basic agent such as sodium hydride in a solvent such as acetonitrile.

According to the invention, the compound of general formula (II) for which Z is a sulfur atom may be prepared starting with a compound of general formula IX as defined above, of which the corresponding thiol is prepared first by preparing the corresponding mesylate of formula (XII)

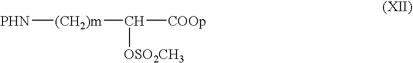

in which P, m and p are defined as above, and then by reacting it with a thioacetate such as cesium or sodium thioacetate, in a solvent such as dimethylformamide, in order to obtain the compound of general formula (XIII)

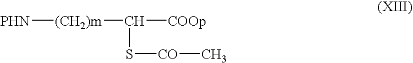

in which P, m and p are defined as above, which is treated with a base, and then the thiol thus obtained is reacted with a compound of formula (X) under the conditions as defined above, in order to obtain a corresponding compound of formula (IIp):

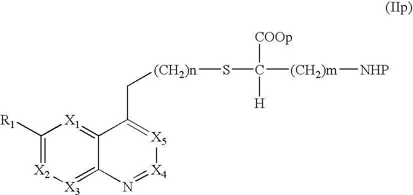

in which P, m, p, n, R₁, X₁, X₂, X₃, X₄ and X₅ are defined as above, which, where appropriate, is deprotected at the level of the amino functional group.

The protecting groups and the methods for introducing them and where appropriate for removing them are those mentioned above.

The preparation of the mesylate of formula (XII) may be carried out in pyridine.

The reaction of the compound of formula (XII) may be carried out in a solvent such as dimethylformamide.

According to the invention, the compound of general formula (II) for which Z is a group NH may be prepared starting with a compound of general formula (XIV)

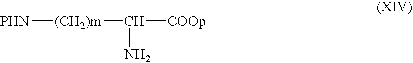

in which P, m and p are defined as above, which is reacted with a compound of formula (X) under the conditions as defined above, in order to obtain a correspondi ng compound of formula (Ip)

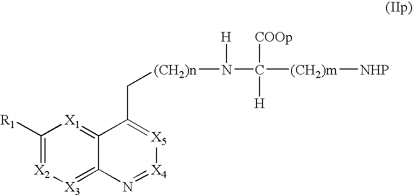

in which P, m, p, n, R₁, X₁, X₂, X₃, X₄ and X₅ are defined as above, which, where appropriate, is deprotected at the level of the amino functional group.

The protecting groups and the methods for introducing them and where appropriate for removing them are those mentioned above.

The compound of formula (XIV) may be prepared starting with a compound of formula

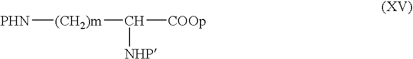

in which P, m and p are defined as above and P′ is a protecting group different from P and removable under different conditions from P.

The compound of formula (XV) may be prepared starting with the corresponding acid. Such acids are known or can be prepared by known methods and for some are commercial products.

The compound of formula (II) as defined above in which R₁, X₁, X₂, X₃, X₄, X₅, Z, n, R₂, R₅, m and R₄ are defined as above and Y is a group CHR in which R is an alkyl radical may be prepared starting with the corresponding compound in which R is a hydrogen atom by preparing the anion at the α position of quinoline under conditions similar to those indicated above for the compound of formula (III′), with which anion a reagent of the RX type is reacted, X being a halogen such as chlorine or, preferably, bromine or iodine or alternatively a leaving group such as a mesyl or a tosyl.

Such a compound may also be prepared starting with a compound in which Y is a group CO, by the action of an appropriate magnesium compound under conditions known to persons skilled in the art, followed where appropriate by deoxygenation under conditions which are also known to persons skilled in the art, in particular which are described by Barton et al., J. Chem. Soc, Perkin trans. 1, 1574 (1975) and Synthesis, 743 (1981) and by N. Hartwig, Tetrahedron, 39, 2609 (1983).

It is understood that the derivatives of general formulae (I) and (II) can exist in enantiomeric or diastereoisomeric forms or in E or Z form, which of course fall within the scope of the present invention. These forms may be separated according to the usual methods, known to persons skilled in the art, in particular by chiral chromatography or by High Performance Liquid Chromatography (HPLC). This is illustrated below in the experimental section.

The derivatives of general formula (I) can be purified, where appropriate, by physical methods such as crystallization or chromatography.

The derivatives of general formula (I) may be, where appropriate, converted to addition salts with acids or with bases by known methods. It is understood that these salts with acids or bases also fall within the scope of the present invention.

As examples of addition salts with pharmaceutically acceptable acids, there may be mentioned the salts formed with inorganic acids (for example hydrochlorides, hydrobromides, sulfates, nitrates or phosphates) or with organic acids (for example succinates, fumarates, tartrates, acetates, propionates, maleates, citrates, methanesulfonates, ethanesulfonates, phenylsufonates, p-toluenesulfonates, isethionates, naphthylsulfonates or camphorsulfonates) or with substitution derivatives of these acids.

The derivatives of general formula (I) carrying a carboxyl radical may be converted to metal salts or to addition salts with nitrogenous bases according to methods known per se. The salts may be obtained by the action of a metal (for example an alkali or alkaline-earth metal) base, of ammonia or of an amine, on a product according to the invention, in an appropriate solvent such as an alcohol, an ether or water, or by an exchange reaction with a salt of an organic acid. The salt formed precipitates after optional concentration of the solution, it is separated by filtration, decantation or lyophilization. As examples of pharmaceutically acceptable salts, there may be mentioned in particular the salts with alkali metals (sodium, potassium, lithium) or with alkaline-earth metals (magnesium, calcium), ammonium salt, the salts of nitrogenous bases (ethanolamine, diethanolamine, trimethylamine, triethylamine, methylamine, propyl-amine, diisopropylamine, N,N-dimethylethanolamine, benzylamine, dicyclohexylamine, N-benzyl-p-phen-ethylamine, N,N′-dibenzylethylenediamine, diphenylene-diamine, benzhydrylamine, quinine, choline, arginine, lysine, leucine, dibenzylamine).

The derivatives of general formula (I) according to the invention are particularly active antibacterial agents.

The study below demonstrates this.

a) Activity in vitro

The method of dilutions in agar medium in agreement with the NCCLS recommendations is used for the determination of the minimum inhibitory concentrations (MIC) expressed in mg/l.

The activities of the compounds of examples 5, 8, 10-13 are grouped together in the following table:

Gram-positive MIC mg/l at 24 hours

| | |
|---|---|
| S. aureus IP8203 sensitive | 0.12-1 |
| S. aureus AS 5155 methicillin resistant | 0.12-1 |
| S. pneumoniae 6254-01 MLS_{B} resistant | 0.25-0.5 |
| E. faecalis ATCC S343211 vancomycin | 0.25-1 |
| resistant | |

Gram-negative MIC mg/l at 48 hours

| | |
|---|---|
| M. catarrhalis IPA151 sensitive | 0.5-4 |
| H. influenzae MDK 1528 sensitive | 2-8 |

In vitro, the compounds of the invention therefore proved quite remarkable both on Gram-positive microorganisms and on Gram-negative microorganisms.

b) The products according to the invention are particularly advantageous because of their low toxicity, most of the products not having exhibited toxicity at the dose of 50 mg/kg (DC50) both by the subcutaneous route and by the oral route in mice (2 administrations/day).

These properties make said products, and their salts with pharmaceutically acceptable acids and bases, suitable for use as medicaments in the treatment of conditions caused by sensitive microorganisms brought about by Gram-positive bacteria and in particular in those caused by staphylococcus, such as staphylococcal septicemia, facial or cutaneous malignant staphylo-coccia, pyoderma, septic or suppurant wounds, anthrax, phlegmons, erysipela, primitive or post-influenza acute staphylococcia, bronchopneumonia, pulmonary suppurations, and in those caused by streptococci or enterococci.

These products may also be used as medicaments in the treatment of upper and lower respiratory infections caused by Gram-negative bacteria such as Haemophilus influenzae and Moraxella catarrhalis.

The subject of the present invention is therefore also, as medicaments and in particular medicaments intended for the treatment of bacterial infections in humans or animals, the compounds of general formula (I) as defined above and their pharmaceutically acceptable salts, in particular the preferred compounds mentioned above.

The present invention also relates to the pharmaceutical compositions containing at least one 4-substituted quinoline derivative according to the invention, where appropriate in salt form, in the pure state or in the form of a combination with one or more compatible and pharmaceutically acceptable diluents or adjuvants.

The compositions according to the invention may be used by the oral, parenteral, topical or rectal route or as aerosols.

As solid compositions for oral administration, there may be used tablets, pills, gelatin capsules, powders or granules. In these compositions, the active product according to the invention is mixed with one or more inert diluents or adjuvants, such as sucrose, lactose or starch. These compositions may comprise substances other than diluents, for example a lubricant such as magnesium stearate or a coating intended for a controlled release.

As liquid compositions for oral administration, there may be used solutions which are pharmaceutically acceptable, suspensions, emulsions, syrups and elixirs containing inert diluents such as water or paraffin oil. These compositions may also comprise substances other than diluents, for example wetting products, sweeteners or flavorings.

The compositions for parenteral administration may be sterile solutions or emulsions. As a solvent or vehicle, it is possible to use water, propylene glycol, a polyethylene glycol, vegetable oils, in particular olive oil, organic esters for injection, for example ethyl oleate. These compositions may also contain adjuvants, in particular wetting, isotonizing, emulsifying, dispersing and stabilizing agents.

The sterilization may be carried out in several ways, for example using a bacteriological filter, by irradiation or by heating. They may also be prepared in the form of sterile solid compositions which may be dissolved at the time of use in sterile water or any other sterile medium for injection.

The compositions for topical administration may be for example creams, ointments, lotions or aerosols.

The compositions for rectal administration are suppositories or rectal capsules which contain, in addition to the active ingredient, excipients such as cocoa butter, semisynthetic glycerides or polyethylene glycols.

The compositions may also be aerosols. For use in the form of liquid aerosols, the compositions may be stable sterile solutions or solid compositions dissolved at the time of use in pyrogen-free sterile water, in saline or any other pharmaceutically acceptable vehicle. For use in the form of dry aerosols intended to be directly inhaled, the active ingredient is finely divided and combined with a water-soluble solid diluent or vehicle having a particle size of 30 to 80 μm, for example dextran, mannitol or lactose.

In human therapy, the novel 4-substituted quinoline derivatives according to the invention are particularly useful in the treatment of infections of bacterial origin. The doses depend on the desired effect and the duration of the treatment. The doctor will determine the dosage which he judges most appropriate according to the treatment, and according to the age, the weight, the degree of the infection and the other factors specific to the subject to be treated. As a guide, the doses may be between 750 mg and 3 g of active product in 2 or 3 doses per day by the oral route or between 400 mg and 1.2 g by the intravenous route for an adult. The following examples illustrate compositions according to the invention.

a) A liquid composition intended for parenteral use is prepared according to the usual technique, comprising:

| | |
|---|---|
| (RS)-2-{[(E)-3-(2,5-difluorophenyl)- | 1 g |
| (allylamino]methyl}-5-(3-fluoro-6- | |
| methoxyquinolin-4-yl)pentanoic acid | |
| Glucose | qs 2.5% |
| Sodium hydroxide | qs pH = 4-4.5 |
| Water for injection | qs 20 ml |

b) A liquid composition intended for parenteral use is prepared according to the usual technique, comprising:

| | |
|---|---|
| (RS)-2-{[2-(2,5-difluorophenyl- | 0.5 g |
| sulfanyl)ethylamino]methyl}-5-(3- | |
| fluoro-6-methoxyquinolin-4-yl)- | |
| pentanoic acid | |
| Glucose | qs 5% |
| Sodium hydroxide | qs pH = 4-4.5 |
| Water for injection | qs 50 ml |

The following examples illustrate the invention.

### EXAMPLE 1

Ethyl (RS)-2-aminomethyl-5-(3-fluoro-6-methoxyquinolin-4-yl)pentanoate hydrochloride may be prepared in the following manner:

1). 88.4 cm³ of a 4N hydrochloric acid solution in dioxane are added at a temperature in the region of 20° C. to 15.37 g (35.37 mmol) of ethyl (RS)-2-tert-butyloxycarbonylaminomethyl)-5-(3-fluoro-6-methoxy-quinolin-4-pentanoate in solution in 268 cm³ of ethanol. After stirring for 15 hours at a temperature in the region of 20° C., the reaction mixture is concentrated to dryness under reduced pressure to give 11.72 g of ethyl (RS)-2-aminomethyl-5-(3-fluoro-6-methoxyquinolin-4-yl)pentanoate hydrochloride in the form of a yellow solid.

EI MS spectrum: m/z 334 [M+], m/z 178 (base peak).

Ethyl (RS)-2-(tert-butyloxycarbonylaminomethyl)-5-(3-fluoro-6-methoxyquinolin-4-yl)pentanoate may be prepared in the following manner:

2). A solution of 12.43 g (48.3 mmol) of ethyl (RS)-2-(tert-butyloxycarbonylaminomethyl)pent-4-enoate in 150 cm³ of tetrahydrofuran is added at a temperature in the region of 0° C., under an argon atmosphere, to 144.9 cm³ (72.45 mmol) of a solution of 0.5M 9-BBN (9-borabicyclo[3.3.1]nonane)/THF. After heating the reaction mixture to a temperature in the region of 20° C. and then stirring for 3.25 hours at a temperature in the region of 20° C., 14.64 g (48.3 mmol) of 3-fluoro-4-iodo-6-methoxyquinoline in suspension in 370 cm³ of tetrahydrofuran and then 30.76 g (145 mmol)of potassium phosphate and 1.06 g (1.449 mmol) of PdCl₂dppf ([1,1′-bis(diphenylphosphino)ferrocene]palladium chloride) are successively added. After stirring for 16 hours at the reflux temperature, the reaction mixture is cooled and then filtered on Celite®. The Celite® is rinsed with tetrahydrofuran. The filtrate is then concentrated to dryness under reduced pressure (2.7 kPa). The residue is taken up in ethyl acetate, washed with water and then with a saturated aqueous sodium chloride solution. The organic phase is dried and concentrated to dryness under reduced pressure (2.7 kPa) to give 30.6 g of a brown oil which is purified by flash chromatography [eluent: cyclohexane/ethyl acetate (7/3 by volume)]. 15.37 g of ethyl (RS)-2-(tert-butyloxycarbonyl-aminomethyl)-5-(3-fluoro-6-methoxyquinolin-4-yl)-pentanoate are obtained in the form of a yellow oil.

EI MS spectrum: m/z 434 [M+], m/z 204 (base peak). 3-Fluoro-4-iodo-6-methoxyquinoline may be prepared according to the method described in patent WO 2002/40474-A2.

Ethyl (RS)-2-(tert-butyloxycarbonylaminomethyl)pent-4-enoate may be prepared in the following manner:

3). 147 cm³ (174 mmol) of lithium bis(trimethylsilyl)-amide in 1M solution in tetrahydrofuran are added dropwise at a temperature in the region of −78° C., under an argon atmosphere, to 18.9 g (87 mmol) of ethyl 3-tert-butyloxycarbonylaminopropionate in solution in 203 cm³ of tetrahydrofuran. After stirring for 0.5 hour at a temperature in the region of −78° C., 7.51 cm³ (87 mmol) of allyl bromide are added. After stirring for 5 hours at a temperature in the region of −78° C., the temperature is allowed to change from −78° C. to a temperature in the region of 20° C. over 1.5 hours. The reaction medium is then hydrolyzed with 150 cm³ of water. The organic phase is separated by decantation, diluted with ethyl acetate, washed with water and with a saturated aqueous sodium chloride solution, dried and then concentrated to dryness under reduced pressure (2.7 kPa) to give 16.7 g of a colorless oil which is purified by flash chromatography [eluent: cyclohexane/ethyl acetate (8/2 by volume)]. 12.43 g of ethyl (RS)-2-(tert-butyloxycarbonylaminomethyl)pent-4-enoate are obtained in the form of a colorless oil.

CI MS spectrum: m/z 258 [M+H]+(base peak).

Ethyl 3-tert-butyloxycarbonylaminopropionate may be prepared in the following manner:

4). 59.9 cm³ or triethylamine and then 46.88 g of di-tert-butyl dicarbonate are successively added at a temperature in the region of 20° C., under an argon atmosphere, to 30 g (195 mmol) of β-alanine ethyl ester hydrochloride in solution in 1000 cm³ of dichloromethane. After stirring for 20 hours at a temperature in the region of 20° C., the reaction mixture is successively washed with twice 500 cm³ of water, twice 500 cm³ of a 0.1N aqueous hydrochloric acid solution and twice 500 cm³ of a saturated aqueous sodium bicarbonate solution. The organic phase is dried and then concentrated to dryness under reduced pressure (2.7 kPa) to give 45.6 g of a colorless oil which is purified by flash chromatography [eluent: cyclohexane/ethyl acetate (8/2 by volume)]. 40.5 g of ethyl 3-tert-butyloxycarbonylaminopropionate are obtained in the form of a colorless oil. EI MS spectrum: m/z 258 [M+H]+(base peak).

### EXAMPLE 2

Ethyl (RS)-2-{[(E)-(2,5-difluorophenyl)allylamino]-methyl}-5-(3-fluoro-6-methoxyquinolin-4-yl)pentanoate 1.5 cm³ of triethylamine and 0.816 g of (E)-2-(2,5-difluorophenyl)propenal in solution in 100 cm³ of diethyl ether are added at a temperature in the region of 0° C., under an argon atmosphere, to 2 g (5.393 mmol) of ethyl (RS)-2-aminomethyl-5-(3-fluoro-6-methoxy-quinolin-4-yl)pentanoate obtained in example 1 in solution in 142 cm³ of diethyl ether. After stirring for 1 hour at room temperature, 0.816 g of magnesium sulfate is added. After stirring for 1 hour at room temperature, the reaction mixture is filtered, the magnesium sulfate is rinsed with diethyl ether and then the filtrate is concentrated to dryness under reduced pressure (2.7 kPa) to give an oil which is diluted with 242 cm³ of ethanol. 0.204 g of sodium borohydride is added to this solution which is cooled to a temperature in the region of 0° C., under an argon atmosphere. After stirring for 15 minutes at a temperature in the region of 0° C. and then for 16 hours at room temperature, the reaction mixture is concentrated to dryness under reduced pressure (2.7 kPa) to give a residue which is diluted with 100 cm³ of ethyl acetate, washed with water and then with a saturated aqueous sodium chloride solution. The organic phase is dried over anhydrous magnesium sulfate, filtered and concentrated to dryness under reduced pressure (2.7 kPa) to give 3.3 g of a residue which is purified by flash chromatography [eluent: dichloromethane/methanol/acetonitril (98/1/1 by volume)]. 1.38 g of ethyl (RS)-2-{[(E)-3-(2,5-difluorophenyl)allylamino]methyl}-5-(3-fluoro-6-methoxyquinolin-4-yl)penanoate are obtained in the form of a pale yellow oil.

EI MS spectrum: m/z 486 [M+.], m/z 153 (base peak).

(E)-3-(2,5-Difluorophenyl)propenal may be prepared in the following manner:

10.6 g of 2,5-difluorobenzaldehyde are added at a temperature in the region of 20° C. to 22.7 g (74.6 mmol) of (triphenylphosphoranylidene)acetaldehyde in solution in 650 cm³ of toluene. After stirring for 4 hours at a temperature in the region of 80° C., the reaction medium is is concentrated to dryness under reduced pressure (2.7 kPa) to give 28.42 g of brown residue which is taken up in 120 cm³ of diisopropyl ether. After stirring for 1 hour at room temperature, the solution is filtered and the solid residue is taken up in 120 cm³ of diisopropyl ether. After stirring for 1.5 hours at room temperature, the solution is filtered and then the two filtrates are combined and concentrated to dryness under reduced pressure (2.7 kPa) to give 11.69 g of a yellow solid which is purified by flash chromatography [eluent: ethyl acetate/cyclohexane (1/1 by volume)]. 9.32 g of a pale yellow solid are obtained, which solid is recrystallized in the hot state from 20 cm³ of diisopropyl ether to give 6.66 g of (E)-3-(2,5-difluorophenyl)propenal in the form of a pale yellow solid melting at 88° C.

MS EI spectrum: m/z 168 [M+].

### EXAMPLE 3 AND EXAMPLE 4

Enantiomers A (levorotatory) and B (dextrorotatory) of ethyl (RS)-2-{[(E)-3-(2,5-difluorophenyl)allylamino]-methyl}-5-(3-fluoro-6-methoxyquinolin-4-yl)pentanoate

The ethyl (RS)-2-{[(E)-3-(2,5-difluorophenyl)-allylamino]methyl}-5-(3-fluoro-6-methoxyquinolin-4-yl)pentanoate (0.750 g) obtained in example 2 in solution in 15 cm³ of ethanol and 60 cm³ of heptane is injected onto a column 8 cm in diameter and 35 cm in length containing 1200 g of chiral stationary phase: Chiralpak ADTM having a particle size of 20 μm. The elution is carried out with a mobile phase [heptane/ethanol/methanol (96/2/2 by ovlume)] at a flow rate of 140 ml/min, the detection is carried out by UV at 254 nm. The enantiomer A (levorotatory), of undetermined absolute configuration, which elutes first, is recovered and then concentrated under reduced pressure (2.7 kPa) at a temperature in the region of 35° C. to give 0.359 g of a colorless oil. The enantiomer B (dextrorotatory), of undetermined absolute configuration, which elutes second, is recovered and then concentrated under reduced pressure (2.7 kPa) at a temperature in the region of 35° C. to give 0.369 g of a colorless oil.

Enantiomer A (levorotatory) of ethyl 2-{[(E)-3-(2,5-difluorophenyl)allylamino]methyl}-5-(3-fluoro-6-methoxyquinolin-4-yl)pentanoate [α]D20 -4.7+/−0.4 [methanol (c=0.5), 589 nm)]. ¹H NMR spectrum (300 MHz, (CD₃)₂SO d6, δin ppm): 1.12 (t, J=7 Hz: 3H); 1.64 (unresolved complex: 4H); 1.99 (broad unresolved complex: 1H); from 2.50 to 2.70 (mt: 2H); 2.74 (mt: 1H); 3.08 (mt: 2H); from 3.20 to 3.35 (mt: 2H); 3.96 (s: 3H); 4.05 (q, J=7 Hz: 2H); 6.44 (dt, J=16 and 5.5 Hz: 1H); 6.59 (broad d, J=16 Hz: 1H); 7.11 (mt: 1H); 7.24 (doublet of t, J=9.5-4.5 Hz: 1H); 7.36 (d, J=3 Hz: 1H); 7.40 (dd, J=9 and 3 Hz: 1H): 7.44 (mt: 1H); 7.97 (d, J=9 Hz: 1H); 8.69 (d, J=0.5 Hz: 1H).

IR spectrum (CCl₄) 2939; 2831; 1729; 1621; 1508; 1491; 1468; 1263; 1231; 1182; 1034; 971 and 832 cm⁻¹.

EI MS spectrum: m/z 486 [M]+., m/z 153 (base peak). Enantiomer B (dextrorotatory) of ethyl 2-{(E)-3-(2,5-difluorophenyl)allylamino]methyl}-5-(3-fluoro-6-methoxyquinolin-4-yl)pentanoate

[α]D20+3.0+/−0.5 [methanol (c=0.5), 589 nm)]. ¹H NMR spectrum (300 MHz, (CD₃)₂SO d6, δ in ppm): 1.11 (t, J=7 Hz: 3H); 1.65 (unresolved complex: 4H); from 2.50 to 2.65 (mt: 2H); 2.75 (dd, J=11 and 8 Hz: 1H); 3.09 (mt: 2H); from 3.20 to 3.35 (mt: 2H); 3.96 (s: 3H); 4.04 (q, J=7 Hz: 2H); 6.44 (dt, J=16 and 5.5 Hz: 1H); 6.60 (broad d, J=16 Hz: 1H); 7.12 (mt: 1H); 7.24 (ddd, J=10-9 and 5 Hz: 1H); 7.36 (d, J=3 Hz: 1H); 7.39 (dd, J=9 and 3 Hz: 1H): 7.45 (mt: 1H); 7.96 (d, J=9Hz: 1H); 8.68 (d, J=0.5 Hz: 1H).

IR spectrum (CC1₄) 2939; 2831; 1729; 1621; 1508; 1491; 1468; 1263; 1231; 1182; 1034; 971 and 832 cm⁻¹.

EI MS spectrum: m/z 486 [M]+., m/z 153 (base peak).

### EXAMPLE 5

(RS)-2-{[(E)-3-(2,5-difluorophenyl)allylamino]methyl}-5-(3-fluoro-6-methoxyquinolin-4-yl)pentanoic acid 7.08 cm³ of a 5N aqueous sodium hydroxide solution are added at a temperature in the region of 20° C. to 0.41 g (0.843 mmol) of ethyl (RS)-2-{[(E)-3-(2,5-difluorophenyl)allylamino]methyl}-5-(3-fluoro-6-methoxyquinolin-4-yl)pentanoate obtained in example 2 in solution in 22 cm³ of dioxane. After stirring under reflux for 20 hours, the reaction medium is concentrated to dryness under reduced pressure (2.7 kPa) to give a pale yellow oil which is purified by flash chromatography [eluent: chloroform/methanol (13/2 by volume)+0.5% of an aqueous solution of ammonia at 20%]. 0.295 g of 2-{[(E)-3-(2,5-difluorophenyl)allylamino]methyl}-5-(3-fluoro-6-methoxyquinolin-4-yl)pentanoic acid is obtained in the form of white solid melting at 130° C.

IR spectrum (KBr) 2942; 1622; 1509; 1491; 1231; 1146; 1030; 979; 831 and 727 cm⁻¹.

¹H NMR spectrum (300 MHz, (CD₃) ₂SO d6, δin ppm): from 1.50 to 1.75 (mt: 4H); 2.42 (mt: 1H); from 2.65 to 2.75 (mt: 2H); 3.08 (mt: 2H); from 3.20 to 3.50 (mt: 2H); 3.96 (s: 3H); 6.48 (dt, J=16 and 6 Hz: 1H); 6.65 (broad d, J=16 Hz: 1H); 7.13 (mt: 1H); 7.24 (doublet of t, J=9.5 and 5 Hz: 1H); 7.38 (mt: 2H); 7.47 (ddd, J=10-6 and 3 Hz: 1H); 7.95 (mt: 1H); 8.68 (broad s: 1H).

ES+MS spectrum: m/z 459 [M+H]+ (base peak).

### EXAMPLE 6

Enantiomer A of 2-{[(E)-3-(2,5-difluorophenyl)-allylamino]methyl}-5-(3-fluoro-6-methoxyquinolin-4-yl)pentanoic acid (undetermined absolute configuration) 6.18 cm³ of a 5N aqueous sodium hydroxide solution are added at a temperature in the region of 20° C. to 0.358 g (0.736 mmol) of enantiomer A (levorotatory) of ethyl 2-{[(E)-3-(2,5-difluorophenyl)allylamino]methyl}-5-(3-fluoro-6-methoxyquinolin-4-yl)pentanoate obtained in example 3 in solution in 20 cm³ of dioxane. After stirring under reflux for 20 hours, the bottom phase is removed and the top phase is concentrated to dryness under reduced pressure (2.7 kPa) to give a residue which is taken up in 5 cm³ of water and 20 cm³ of dichloromethane. The aqueous phase is acidified with 1N hydrochloric acid to a pH value in the region of 7. The reaction mixture is concentrated to dryness under reduced pressure (2.7 kPa) to give a residue which is taken up in 3 cm³ of water and 15 cm³ of acetonitrile. After stirring for 2 hours at room temperature, the reaction mixture is filtered. The residue is washed with acetonitrile and then dried under reduced pressure (2.7 kPa) at a temperature in the region of 35° C. for 2 hours to give 0.293 g of a white solid which is purified by flash chromatography [eluent: chloroform/methanol (12/3 by volume)+0.5% of an aqueous solution of ammonia at 20%]. A white solid is obtained which is triturated in a mixture of 9 cm³ of acetonitrile and 1 cm³ of water. After filtration, the white solid is dried under reduced pressure (2.7 kPa) at a temperature in the region 35° C. for 16 hours to give 0.158 g of enantiomer A of 2-{[(E)-3-(2,5-difluorophenyl)allylamino]methyl}-5-(3-fluoro-6-methoxyquinolin-4-yl)pentanoic acid (undetermined absolute configuration) in the form of a white solid melting at 168° C; [α]D20 0+/−0.4 [dimethyl sulfoxide (c=0.5,), 589 nm)].

¹H NMR spectrum (300 MHz, (CD₃) ₂SO d6, δin ppm) : from 1.50 to 1.75 (mt: 4H); from 2.40 to 2.60 (mt: 1H); from 2.65 to 2.80 (mt: 2H); 3.08 (mt: 2H); from 3.20 to 3.50 (mt: 2H); 3.97 (broad s: 3H); 6.47 (dmt, J=16 Hz: 1H); 6.64 (broad d, J=16 Hz: 1H); 7.13 (mt: 1H); 7.24 (mt: 1H); from 7.30 to 7.50 (mt: 3H); 8.06 (broad d, J=9 Hz: 1H); 8.68 (broad s, 1H).

IR spectrum (KBr) 2940; 1648; 1621; 1592; 1509; 1492; 1432; 1363; 1234; 1148; 1029; 988; 831; 791 and 728 cm¹.

EI MS spectrum: m/z 458 [M]+., m/z 153 (base peak).

### EXAMPLE 7

Enantiomer B of 2-{[(E)-3-(2,5-difluorophenyl)allyl-amino]methyl}-5-(3-fluoro-6-methoxyquinolin-4-yl)pentanoic acid (undetermined absolute configuration) 6.08 cm³ of a 5N aqueous sodium hydroxide solution are added at a temperature in the region of 20° C. to 0.352 g (0.724 mmol) of enantiomer B (dextrorotatory) of ethyl 2-{[(E)-3-(2,5-difluorophenyl)allylamino]methyl}-5-(3-fluoro-6-methoxyquinolin-4-yl)pentanoate obtained in example 4 in solution in 20 cm³ of dioxane. After stirring under reflux for 20 hours, the bottom phase is removed and the top phase is concentrated to dryness under reduced pressure (2.7 kPa) to give a residue which is taken up in 5 cm³ of water and 20 cm³ of dichloromethane. The aqueous phase is acidified with iN hydrochloric acid to a pH value in the region of 7. The reaction mixture is concentrated to dryness under reduced pressure (2.7 kPa) to give a residue which is purified by 2 successive flash chromatographies [eluent: chloroform/methanol (12/3 by volume)+0.5% of an aqueous solution of ammonia at 20%]. A white solid is obtained which is triturated in a mixture of 9 cm³ of acetonitrile and 1 cm³ of water. After filtration, the white solid is dried under reduced pressure (2.7 kPa) at a temperature in the region 35° C. for 16 hours to give 0.136 g of enantiomer B of 2-{[(E)-3-(2,5-difluorophenyl)allylamino]methyl}-5-(3-fluoro-6-methoxyquinolin-4-yl)pentanoic acid (undetermined absolute configuration) in the form of a white solid melting at 166° C.; [α]D20 0 −3.4+/−0.4 [dimethyl sulfoxide (c=0.5,), 589 nm)].

¹H NMR spectrum (300 MHz, (CD₃) 2SO d6, δ in ppm): from 1.50 to 1.75 (mt: 4H); 2.41 (mt: 1H); from 2.65 to 2.75 (mt: 2H); 3.07 (mt: 2H); from 3.20 to 3.50 (mt: 2H); 3.96 (s: 3H); 6.47 (dt, J=16 Hz and 6 Hz: 1H); 6.65 (broad d, J=16 Hz: 1H); 7.13 (mt: 1H); 7.25 (doublet of t, J=9.5-4.5 Hz: 1H); 7.39 (mt: 2H); 7.47 (ddd, J=9.5-6 and 3 Hz: 1H); 7.96 (mt: 1H); 8.68 (broad s: 1H).

IR spectrum (KBr) 2940; 1648; 1621; 1592; 1509; 1492; 1432; 1363; 1234; 1148; 1029; 988; 831; 791 and 728 cm⁻¹.

EI MS spectrum: m/z 458 [M]+., m/z 153 (base peak).

### EXAMPLE 8

(RS)-2-{[3-(2,5-difluorophenyl)propylamino]methyl}-5-(3-fluoro-6-methoxyquinolin-4-yl)pentanoic acid 6.88 cm³ of a 5N aqueous sodium hydroxide solution are added at a temperature in the region of 20° C. to 0.4 g (0.819 mmol) of ethyl (RS)-2-{[3-(2,5-difluorophenyl)-propylamino]methyl}-5-(3-fluoro-6-methoxyquinolin-4-yl)pentanoate obtained in solution in 22 cm³ of dioxane. After stirring under reflux for 20 hours, the bottom phase is removed and the top phase is concentrated to dryness under reduced pressure (2.7 kPa) to give 0.42 g of a yellow oil which is purified by flash chromatography [eluent: chloroform/methanol (12/3 by volume)+0.5% of an aqueous solution of ammonia at 20%]. 0.344 g of (RS)-2-{[3-(2,5-difluorophenyl)propylamino]methyl}-5-(3-fluoro-6-methoxyquinolin-4-yl)pentanoic acid is obtained in the form of a white solid melting at 186° C.

IR spectrum (KBr) 2947; 1645; 1621; 1509; 1496; 1470; 1229; 1140; 1033; 833; 789 and 721 cm⁻¹.

ES+MS spectrum: m/z 461 [M+H]+(base peak).

¹H NMR spectrum (300 MHz, (CD₃)₂SO d6, δ in ppm): 1.54 (mt: 1H); from 1.60 to 1.85 (mt: 5H); 2.30 (mt: 1H); from 2.55 to 2.85 (mt: 6H); 3.08 (mt: 2H); 3.97 (s: 3H); 7.10 (mt: 1H); from 7.15 to 7.25 (mt: 2H); 7.38 (mt: 2H); 7.96 (mt: 1H); 8.69 (broad s: 1H).

Ethyl (RS)-2-{[3-(2,5-difluorophenyl)propylamino]-methyl}-5-(3-fluoro-6-methoxyquinolin-4-yl)pentanoate may be prepared in the following manner:

31 cm³ of ethanol and 0.4 g (0.822 mmol) of ethyl (RS)-2-{[(E)-3-(2,5-difluorophenyl)allylamino]methyl}-5-(3-fluoro-6-methoxyquinolin-4-yl)pentanoate are added at room temperature, under an argon atmosphere, to 0.043 g (0.405 mmol) of 10% palladium on carbon. The reaction medium is purged 5 times with argon and then hydrogenated at a pressure of 2 bar of hydrogen at room temperature for 6 h. The catalyst is filtered on Celite®, the Celite® is rinsed with 3 times 5 cm³ of ethanol and then the filtrate is concentrated to dryness under reduced pressure (2.7 kPa) to give 0.459 g of ethyl (RS)-2-{[3-(2,5-difluorophenyl)-propylamino]methyl}-5-(3-fluoro-6-methoxyquinolin-4-yl)pentanoate in the form of a colorless oil.

EI MS spectrum: m/z 488 [(M+.], m/z 204 (base peak).

### EXAMPLE 9

Ethyl (RS)-2-({N-[(E)-3-(2,5-difluorophenyl)allyl]-N-methylamino}methyl)-5-(3-fluoro-6-methoxyquinolin-4-yl)pentanoate may be prepared in the following manner:

0.988 g (32.9 mmol) of formaldehyde is added at a temperature in the region of 0° C., under an argon atmosphere, to 0.87 g (1.788 mmol) of ethyl (RS)-2-{[(E)-3-(2,5-difluorophenyl)allylamino]methyl}-5-(3-fluoro-6-methoxyquinolin-4-yl)pentanoate obtained in example 2 in solution in 200 cm³ of ethanol. After stirring for 0.25 hour at a temperature in the region of 0° C., 1.516 g (7.15 mmol) of sodium triacetoxy-borohydride are added. After stirring for 20 hours at room temperature, 0.494 g of formaldehyde and 0.758 g of sodium triacetoxyborohydride are again added. After stirring for 3 hours at room temperature, the reaction mixture is concentrated to dryness under reduced pressure (2.7 kPa), diluted with 50 cm³ of ethyl acetate and then washed with water and then with a saturated aqueous sodium chloride solution. The organic phase is dried, filtered and concentrated to dryness under reduced pressure (2.7 kPa) to give 0.778 g of ethyl (RS)-2-({N-[(E)-3-(2,5-difluorophenyl)allyl]-N-methylamino}methyl)-5-(3-fluoro-6-methoxyquinolin-4-yl)pentanoate in the form of a white oil.

EI MS spectrum: m/z 500 [M+.], m/z 153 (base peak).

### EXAMPLE 10

Sodium (RS)-2-({N-[(E)-3-(2,5-difluorophenyl)allyl]-N-methylamino}methyl)-5-(3-fluoro-6-methoxyquinolin-4-yl)pentanoate 13.05 cm³ of a 5N aqueous sodium hydroxide solution are added at a temperature in the region of 20° C. to 0.778 g (1.554 mmol) of ethyl (RS)-2-({N-[(E)-3-(2,5-difluoro-phenyl)allyl]-N-methylamino}methyl)-5-(3-fluoro-6-methoxyquinolin-4-yl)pentanoate obtained in example 9 in solution in 40 cm³ of dioxane. After stirring under reflux for 16 hours, the bottom phase is removed and the top phase is concentrated to dryness under reduced pressure (2.7 kPa) to give a residue which is taken up in 40 cm³ of ethyl acetate and 10 cm³ of water. The pH of the aqueous phase is adjusted to 1 by adding a 1N aqueous hydrochloric acid solution. The organic phase is separated by decantation, washed with water and then with a saturated aqueous sodium chloride solution, dried, filtered and then concentrated to dryness under reduced pressure (2.7 kPa) to give 0.625 g of sodium (RS)-2-({N-[(E)-3-(2,5-difluorophenyl)allyl]-N-methyl-amino}methyl)-5-(3-fluoro-6-methoxyquinolin-4-yl)pentanoate in the form of a white solid melting between 60-70° C.

IR spectrum (KBr) 2945; 1621; 1590; 1509; 1490; 1468; 1428; 1231; 1145; 1030; 974; 830 and 727 cm⁻¹.

¹H NMR spectrum (300 MHz, (CD₃)₂SO d6, δ in ppm): from 1.50 to 1.75 (mt: 4H); 2.16 (broad s: 3 H); 2.32 (mt: 1H); from 2.35 to 2.60 (mt: 2H); 3.04 (mt: 2H); 3.13 (broad d, J=5.5 Hz: 2H); 3.95 (s: 3H); 6.40 (dt, J=16 and 5.5 Hz: 1H); 6.59 (broad d, J=16 Hz: 1H); 7.11 (mt: 1H); 7.23 (broad doublet of t, J=9.5 and 6 Hz: 1H); 7.36 (broad d, J=9: 1H); 7.37 (broad s: 1H); 7.45 (mt: 1H); 7.94 (broad d, J=9 Hz: 1H); 8.65 (broad s: 1H).

ES+MS spectrum: m/z 473 {M+H]+(base peak).

### EXAMPLE 11

(RS)-5-(3-Fluoro-6-methoxyquinolin-4-yl)-2-{[2-(thiophen-2-ylsulfanyl)ethylamino]methyl}pentanoic acid 5.28 cm³ of a 5N aqueous sodium hydroxide solution are added at a temperature in the region of 20° C. to 0.3 g (0.629 mmol) of ethyl (RS)-5-(3-fluoro-6-methoxy-quinolin-4-yl)-2-{[2-(thiophen-2-ylsulfanyl)ethyl-amino]methyl}pentanoate in solution in 22 cm³ of dioxane. After stirring under reflux for 20 hours, the bottom phase is removed and the top phase is concentrated to dryness under reduced pressure (2.7 kPa) to give a residue which is taken up in 20 cm³ of dichloromethane and in a 1N aqueous hydrochloric acid solution qs pH=7. The organic phase is separated by decantation and the aqueous phase is extracted with dichloromethane. The organic phases are combined, dried and then concentrated to dryness under reduced pressure to give a white solid which is stirred in 8 cm³ of acetonitrile at a temperature in the region of 0° C. After filtration and drying in an oven under reduced pressure (2.7 kPa) at a temperature in the region of 35° C. for 4 hours, 0.252 g of (RS)-5-(3-fluoro-6-methoxyquinolin-4-yl)-2-{[2-(thiophen-2-ylsulfanyl)-ethylamino]methyl}pentanoic acid is obtained in the form of a white solid melting at 170° C.

IR spectrum (KBr) 2951; 1649; 1510; 1468; 1400; 1361; 1225; 1031; 847; 831; 785; 702 and 691 cm⁻¹.

ES+MS spectrum: m/z 449 {M+H]+(base peak).

¹H NMR spectrum (300 MHz, (CD₃)₂SO d6, δ in ppm): from 1.55 to 1.80 (mt: 4H); 2.39 (mt: 1H); from 2.60 to 2.80 (mt: 4H); 2.88 (t, J=6.5 Hz: 2H); 3.17 (mt: 2H); 3.97 (s: 3H); 7.05 (dd, J=3.5 and 5.5 Hz: 1H); 7.21 (broad d, J=3.5 Hz: 1H); 7.38 (broad s: 1H); 7.40 (broad d, J=9 Hz: 1H); 7.61 (d, J=5.5 Hz: 1H); 7.98 (d, J=9 Hz: 1H); 8.69 (broad s: 1H).

Ethyl (RS)-5-(3-fluoro-6-methoxyquinolin-4-yl)-2-{[2-(thiophen-2-ylsulfanyl)ethylamino]methyl}pentanoate may be prepared in the following manner.

0.686 g (3.237 mmol) of sodium triacetoxyborohydride is added at a temperature in the region of 15° C., under an argon atmosphere, to 0.4 g (1.079 mmol) of ethyl (RS)-2-aminomethyl-5-(3-fluoro-6-methoxyquinolin-4-yl)-pentanoate hydrochloride obtained in example 1 and 0.303 cm³ of triethylamine (2.158 mmol) in solution in 15 cm³ of dichloromethane, followed dropwise by a freshly prepared solution of (thiophen-2-ylsulfanyl)acetaldehyde (1.079 mmol) in toluene. After stirring for 2 hours at room temperature, 4 cm³ of water are added. The organic phase is separated by decantation, washed with water and then with a saturated aqueous sodium chloride solution, dried and concentrated to dryness under reduced pressure (2.7 kPa) to give 0.529 g of a pale yellow oil which is purified by flash chromatography [eluent: dichloromethane/methanol/acetonitrile (96/2/2 by volume)]. 0.3 g of ethyl (RS)-5-(3-fluoro-6-methoxyquinolin-4-yl)-2-{[2-(thiophen-2-ylsulfanyl)-ethylamino]methyl}pentanoate is obtained in the form of a pale yellow oil.

ES+MS spectrum: m/z 477 [M+H]+(base peak).

The solution of (thiophen-2-ylsulfanyl)acetaldehyde (1.079 mmol) in toluene may be prepared as follows.

0.188 cm³ (1.079 mmol) of N,N-diisopropylethylamine is added at a temperature in the region of 15° C., under an argon atmosphere, to 0.106 cm³ (1.079 mmol) of 2-thiophenethiol in solution in 4 cm³ of toluene. After stirring for 0.5 hour at room temperature, the reaction medium is cooled to a temperature in the region of 5° C. and 0.167 cm³ (1.316 mmol) of a 50% aqueous chloroacetaldehyde solution is added. After stirring for 1 hour at room temperature, the organic phase is separated by decantation, washed with twice 5 cm³ of water, dried over anhydrous magnesium sulfate, filtered and then used immediately as it is in the next step.

Ethyl (RS)-2-aminomethyl-5-(3-fluoro-6-methoxyquinolin-4-yl)pentanoate may be prepared as described in example 6.

### EXAMPLE 12

(RS)-2-{[2-(2,5-Difluorophenylsulfanyl)ethylamino]-methyl}-5-(3-fluoro-6-methoxyquinolin-4-yl)pentanoic acid 3.6 cm³ of a 5N aqueous sodium hydroxide solution are added at a temperature in the region of 20° C. to 0.31 g (0.612 mmol) of ethyl (RS)-2-{[2-(2,5-difluoro-phenylsulfanyl)ethylamino]methyl}-5-(3-fluoro-6-methoxyquinolin-4-yl)pentanoate in solution in 6.2 cm³ of dioxane and 6.2 cm³ of methanol. After stirring under reflux for 18 hours, the reaction medium is concentrated to dryness under reduced pressure (2.7 kpa) to give a residue which is purified by flash chromatography [eluent: dichloromethane/methanol (90/10 and then 80/20 by volume)]. 0.26 g of a residue is obtained which is triturated in 50 cm³ of ethyl ether for 18 hours at room temperature. After filtration, washing of the solid with successively 10 cm³ of ethyl ether and 3 times 10 cm³ of pentane and then drying, 0.273 g of (RS)-2-{[2-(2,5-difluorophenylsulfanyl)-ethylamino]methyl}-5-(3-fluoro-6-methoxyquinolin-4-yl)pentanoic acid is obtained in the form of a white solid melting between 182-187° C.

IR spectrum (KBr) 2946; 1643; 1620; 1578; 1509; 1483; 1403; 1229; 1189; 1032; 907; 832 and 757 cm⁻¹.

ES+MS spectrum: m/z 479 [M+H]+(base peak).

¹H NMR spectrum (300 MHz, (CD₃)₂SO d6, δ in ppm): from 1.50 to 1.75 (mt: 4H); 2.42 (mt: 1H); from 2.60 to 2.90 (mt: 4H); 3.06 (mt: 2H); 3.10 (t, J=6.5 Hz: 2H); 3.96 (s: 3H); 7.09 (mt: 1H); from 7.20 to 7.40 (mt: 2H); 7.38 (broad s: 1H); 7.40 (dd, J=9 and 2.5 Hz: 1H); 7.97 (d, J=9 Hz: 1H); 8.69 (broad s: 1H). Ethyl (RS)-2-{[2-(2,5-difluorophenylsulfanyl)ethyl-amino]methyl}-5-(3-fluoro-6-methoxyquinolin-4-yl)pentanoate: 0.377 g (1.49 mmol) of 2-(2-bromoethylsulfanyl)-1,4-difluorobenzene in solution in 10 cm³ of acetonitrile and then 0.746 g (5.4 mmol) of potassium carbonate and 0.247 g (1.49 mmol) of potassium iodide are added at a temperature in the region of 20° C., under an argon atmosphere, to 0.5 g (1.35 mmol) of ethyl (RS)-2-aminomethyl-3-(3-fluoro-6-methoxyquinolin-4-yl)pentanoate hydrochloride obtained in example 1 in solution in 15 cm³ of acetonitrile. After stirring under reflux for 17 hours, the reaction mixture is concentrated to dryness under reduced pressure (2.7 kPa) to give a residue which is taken up in 25 cm³ of ethyl acetate and washed with water. The organic phase is dried over anhydrous magnesium sulfate, filtered and concentrated to dryness under reduced pressure (2.7 kPa) to give a residue which is purified by flash chromatography [eluent: cyclohexane/ethyl acetate (7/3 and then 5/5 by volume)]. 0.34 g of ethyl (RS)-2-{[2-(2,5-difluorophenylsulfanyl)ethylamino]methyl}-5-(3-fluoro-6-methoxyquinolin-4-yl)pentanoate is obtained in the form of a yellow oil.

ES+MS spectrum: m/z 507 [M+H]+(base peak).

1-(2-Bromoethylsulfanyl)(2,5-difluoro)benzene may be prepared according to the method described in patent application WO 2002/40474.

### EXAMPLE 13

(RS)-2-{[2-(2,5-Difluorophenoxy)ethylamino]methyl}-5-(3-fluoro-6-methoxyquinolin-4-yl)pentanoic acid 5.2 cm³ of a 5N aqueous sodium hydroxide solution are added at a temperature in the region of 20° C. to 0.43 g (0.877 mmol) of ethyl (RS)-2-{[2-(2,5-difluorophenoxy)-ethylamino]methyl}-5-(3-fluoro-6-methoxyquinolin-4-yl)pentanoate in solution in 10 cm³ of dioxane and 10 cm³ of methanol. After stirring under reflux for 20 hours, the reaction medium is concentrated to dryness under reduced pressure (2.7 kPa) to give a residue which is purified by flash chromatography [eluent: chloroform/methanol (12/3 by volume)+0.5% of an aqueous solution of ammonia at 20%]. 0.37 g of (RS)-2-{[2-(2,5-difluorophenoxy)ethylamino]methyl}-5-(3-fluoro-6-methoxyquinolin-4-yl)pentanoic acid is obtained in the form of a white solid melting at 179° C.

IR spectrum (KBr) 2961; 1622; 1567; 1513; 1472; 1409; 1321; 1229; 1204; 1156; 1102; 1030; 950; 901; 852; 802; 783; 718 and 699 cm⁻¹.

ES+MS spectrum: m/z 463 [M+H]+(base peak).

¹H NMR spectrum (300 MHz, (CD₃)₂SO d6, δ in ppm) : from 1.45 to 1.75 (mt: 4H); 2.36 (mt: 1H); from 2.60 to 2.80 (mt: 2H); 2.93 (t, J=5.5 Hz: 2H); 3.06 (mt: 2H); 3.97 (s: 3H); 4.11 (t, J=5.5 Hz: 2H); 6.76 (mt: 1H); 7.12 (ddd, J=10.5-6.5 and 3 Hz: 1H); 7.24 (ddd, J=10.5-9.0 and 5.5 Hz: 1H); 7.39 (dd, J=9 and 2.5 Hz: 1H); 7.41 (broad s: 1H); 7.96 (d, J=9 Hz: 1H); 8.69 (broad s: 1H).

Ethyl (RS)-2-{[2-(2,5-difluorophenoxy)ethylamino]-methyl}-5-(3-fluoro-6-methoxyquinolin-4-yl)pentanoate may be prepared in the following manner:

0.39 g (1.65 mmol) of 2-(2-bromoethoxy)-1,4-difluorobenzene in solution in 10 cm³ of acetonitrile and then 0.83 g (6 mmol) of potassium carbonate and 0.27 g (1.65 mmol) of potassium iodide are added at a temperature in the region of 20° C., under an argon atmosphere, to 0.56 g (1.5 mmol) of ethyl (RS)-2-amino-methyl-5-(3-fluoro-6-methoxyquinolin-4-yl)pentanoate hydrochloride obtained in example 1 in solution in 20 cm³ of acetonitrile. After stirring under reflux for 20 hours, the reaction mixture is cooled to room temperature and then poured over 20 cm³ of water and 30 cm³ of ethyl acetate. The aqueous phase is separated by decantation, saturated with sodium chloride and then extracted with 3 times 30 cm³ of ethyl acetate. The organic phases are combined, dried over anhydrous magnesium sulfate, filtered and concentrated to dryness under reduced pressure (2.7 kPa) to give 0.74 g of a brown oil which is purified by flash chromatography [eluent: ethyl acetate/cyclohexane (9/1 by volume)]. 0.43 g of ethyl (RS)-2-{[2-(2,5-difluorophenoxy)ethyl-amino]methyl}-5-(3-fluoro-6-methoxyquinolin-4-yl)-pentanoate is obtained in the form of a yellow oil.

ES+MS spectrum: m/z 491 [M+H]+(base peak).

2-(2-Bromoethoxy)-1,4-difluorobenzene may be prepared according to the method described in patent application WO 2002/40474.

### EXAMPLE 14

(RS)-2-{[N-[(E)-3-(2,5-difluorophenyl)allyl]-N-(2-fluoroethyl)amino]methyl}-5-(3-fluoro-6-methoxy-quinolin-4-yl)pentanoic acid 6.1 cm3 of a 5N aqueous sodium hydroxide solution are added at a temperature in the region of 20° C. to 0.387 g (0.727 mmol) of ethyl (RS)-2-{[N-[(E)-3-(2,5-difluoro-phenyl)allyl]-N-(2-fluoroethyl)amino]methyl}-5-(3-fluoro-6-methoxyquinolin-4-yl)pentanoate in solution in 20 cm³ of dioxane. After stirring under reflux for 20 hours, the bottom phase is removed and the top phase is concentrated to dryness under reduced pressure (2.7 kPa) to give 0.297 g of a residue which is purified by flash chromatography [eluent: dichloromethane/acetonitrile/methanol (94/3/3 by volume and then 90/5/5 by volume with 0.2% of an aqueous solution of ammonia at 20%). 0.146 g of (RS)-2-{[N-[(E)-3-(2,5-difluorophenyl)allyl]-N-(2-fluoro-ethyl)amino]methyl}-5-(3-fluoro-6-methoxyquinolin-4-yl)pentanoic acid is obtained in the form of a white solid melting at 120° C.

¹H NMR spectrum (400 MHz, (CD₃)₂SO d6, δ in ppm) : from 1.55 to 1.75 (mt: 4H); from 2.45 to 2.70 (mt: 2H); from 2.70 to 2.95 (mt: 3H); 3.08 (mt: 2H); from 3.25 to 3.45 (mt: 2H); 3.96 (s: 3H); 4.49 (dt, J=47 and 5.5 Hz: 2H); 6.44 (dt, J=16 and 6.5 Hz: 1H); 6.65 (broad d, J=16 Hz: 1H); 7.13 (mt: 1H); 7.24 (doublet of t, J=9.5 and 5 Hz: 1H); 7.37 (d, J=3 Hz: 1H); 7.40 (dd, J=9 and 3 Hz: 1H); 7.47 (ddd, J=10-6 and 3 Hz: 1H); 7.96 (d, J=9 Hz: 1H); 8.68 (broad s: 1H).

IR spectrum (CC1₄) 2957; 2831; 1709; 1622; 1509; 1491; 1468; 1429; 1232; 1033; 973 and 832 cm⁻¹.

MS DCI spectrum m/z=505 [MH]+(base peak).

Ethyl (RS)-2-{[N-[(E)-3-(2,5-difluorophenyl)allyl]-N-(2-fluoroethyl)amino]methyl}-5-(3-fluoro-6-methoxy-quinolin-4-yl)pentanoate may be prepared in the following manner:

0.781 g (5.65 mmol) of potassium carbonate, 0.188 g (1.13 mmol) of potassium iodide and 2.08 g (16.38 mmol) of 1-bromo-2-fluoroethane are added at a temperature in the region of 20° C., under an argon atmosphere, to 0.55 g (1.13 mmol) of ethyl (RS)-2-{[(E)-3-(2,5-difluorophenyl)allylamino]methyl}-5-(3-fluoro-6-methoxyquinolin-4-yl)pentanoate obtained in example 2 in solution in 30 cm³ of acetonitrile. After stirring under reflux for 24 hours, 2.08 g (16.38 mmol) of 1-bromo-2-fluoroethane are again added. After stirring under reflux for another 24 hours, the reaction mixture is cooled to room temperature. 30 cm³ of water and 20 cm³ of ethyl acetate are added. The organic phase is separated by decantation, washed successively with water and with a saturated aqueous sodium chloride solution, dried and concentrated to dryness under reduced pressure (2.7 kPa) to give 0.72 g of an orange-colored oil which is purified by flash chromatography [eluent: dichloromethane/acetonitrile/methanol (98/1/1 by volume)]. 0.387 g of ethyl (RS)-2-{[N-[(E)-3-(2,5-difluorophenyl)allyl]-N-(2-fluoroethyl)amino]methyl}-5-(3-fluoro-6-methoxyquinolin-4-yl)pentanoate is obtained in the form of a yellow oil.

ES+MS spectrum m/z=533 [MH]+(base peak).

### EXAMPLE 15

(RS)-2-{[N-[(E)-3-(2,5-Difluorophenyl)allyl]-N-(2-hydroxyethyl)amino]methyl}-5-(3-fluoro-6-methoxy-quinolin-4-yl)pentanoic acid 3.1 cm³ of a 5N aqueous sodium hydroxide solution are added at a temperature in the region of 20° C. to 0.278 g (0.524 mmol) of ethyl (RS)-2-{[N-[(E)-3-(2,5-difluorophenyl)allyl]-N-(2-hydroxyethyl)amino]methyl}-5-(3-fluoro-6-methoxyquinolin-4-yl)pentanoate in solution in 5.6 cm³ of dioxane and 5.6 cm³ of ethanol. After stirring under reflux for 20 hours, the reaction medium is concentrated to dryness under reduced pressure (2.7 kPa) to give a residue which is purified by flash chromatography [eluent: dichloromethane-methanol (gradient 100/0 to 70/30 by volume). 0.132 g of a residue is obtained which is triturated in a volume of 10 cm³ of isopropyl ether and 10 cm³ of pentane for 0.5 hour. After filtration, washing of the solid with pentane and then drying, 0.099 g of (RS)-2-{[N-[(E)-3-(2,5-difluorophenyl)allyl]-N-(2-hydroxyethyl)amino]methyl}-5-(3-fluoro-6-methoxy-quinolin-4-yl)pentanoic acid is obtained in the form of a yellow solid melting at 57° C.

¹H NMR spectrum (300 MHz, (CD₃)₂SO d6 with addition of a few drops of CD3COOD d4, 6 in ppm): from 1.50 to 1.75 (mt: 4H); from 2.60 to 2.85 (mt: 4H); from 2.80 to 2.95 (mt: 1H); 3.06 (mt: 2H); from 3.35 to 3.55 (mt: 2H); 3.54 (t, J=6 Hz: 2H); 3.93 (s: 3H); 6.42 (dt, J=16 and 6.5 Hz: 1H); 6.70 (broad d, J=16 Hz: 1H); from 7.00 to 7.25 (mt: 2H); from 7.25 to 7.45 (mt: 3H); 7.96 (d, J=9 Hz: 1H); 8.64 (broad s: 1H).

IR spectrum (KBr) 3070; 2938; 2869; 1710; 1621; 1510; 1490; 1469; 1429; 1361; 1231; 1145; 1028; 975; 830 and 726 cm⁻¹.

CI MS spectrum: m/z 503 [M+H]+(base peak).

Ethyl (RS)-2-{[N-[(E)-3-(2,5-difluorophenyl)allyl]-N-(2-hydroxyethyl)amino]methyl}-5-(3-fluoro-6-methoxy-quinolin-4-yl)pentanoate may be prepared in the following manner:

0.69 g (5 mmol) of potassium carbonate and 0.12 cm³ (1.5 mmol) of iodoethanol are added at a temperature in the region of 20° C., under an argon atmosphere, to 0.486 g (1 mmol) of ethyl (RS)-2-{[(E)-3-(2,5-difluorophenyl)allylamino]methyl}-5-(3-fluoro-6-methoxyquinolin-4-yl)pentanoate obtained in example 2 in solution in 25 cm³ of acetonitrile. After stirring under reflux for 20 hours, 0.12 cm³ (1.5 mmol) of iodoethanol is again added. After stirring under reflux for another 20 hours, 2 cm³ of iodoethanol are again added. After stirring under reflux for 7 hours, the reaction mixture is cooled to room temperature and then filtered. The residue is washed with acetonitrile. The filtrate is concentrated to dryness under reduced pressure (2.7 kPa) to give a residue which is purified by flash chromatography [eluent: dichloromethane and then ethyl acetate/cyclohexane (7/3 by volume)]. 0.305 g of ethyl (RS)-2-{[N-[(E)-3-(2,5-difluoro-phenyl)allyl]-N-(2-hydroxyethyl)amino]methyl}-5-(3-fluoro-6-methoxyquinolin-4-yl)pentanoate is obtained in the form of a yellow oil.

ES+MS spectrum m/z 531 [M+H]+(base peak).

1. A 4-substituted quinoline derivative, which corresponds to general formula I 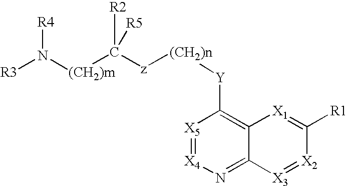
in which:
X₁, X₂, X₃, X₄ and X₅ represent >C-R′₁, to >C-R′₅ respectively, or alternatively at most one of them represents a nitrogen atom,
R₁, R′₁, R′₂, R′₃, R′₄ and R′₅ are identical or different and represent a hydrogen or halogen atom or an alkyl, cycloalkyl, phenyl, phenylthio, mono- or bicyclic heteroaryl or heteroarylthio, OH, SH, alkyloxy, difluoromethoxy, trifluoromethoxy, alkylthio, trifluoromethylthio, cycloalkyloxy, cycloalkylthio, acyl, acyloxy, acylthio, cyano, carboxyl, alkyloxycarbonyl, cycloalkyloxycarbonyl, nitro, —NRaRb or —CONRaRb radical (for which Ra and Rb can represent a hydrogen atom, an alkyl, cycloalkyl, phenyl, mono- or bicyclic heteroaryl radical or Ra and Rb form together with the nitrogen atom to which they are attached a 5- or 6-membered heterocycle which may optionally contain another heteroatom chosen from O, S or N and carrying, where appropriate, an alkyl, phenyl or mono- or bicyclic heteroaryl substituent on the nitrogen atom or, where appropriate, in which the sulfur atom is oxidized to the sulfinyl or sulfonyl state), or represent a methylene radical substituted with fluoro, hydroxyl, alkyloxy, alkylthio, cycloalkyloxy, cycloalkylthio, phenyl, mono- or bicyclic heteroaryl, carboxyl, alkyloxycarbonyl, cycloalkyloxycarbonyl, —NRaRb or —CONRaRb for which Ra and Rb are as defined above, or represent phenoxy, heterocyclyloxy, benzyloxy, heterocyclylmethyloxy,
or alternatively R₁ may also represent difluoromethoxy, or a radical having the structure—Cₘ,F₂ₘ₊₁, -SCₘ, F_{2m′+1} or -OCₘ, F_{2m′+1} for which m′ is an integer from 1 to 6 or alternatively R′₅ may also represent trifluoroacetyl;
n is equal to 0, 1 or 2;
m is equal to 0, 1 or 2;
Y represents a group CHR, C=O, CROH, CRNH₂, CRF or CF₂, R being a hydrogen atom or a (C₁₋₆) alkyl radical;
Z represents a group CH₂ or alternatively Z represents an oxygen atom, a sulfur atom or a group NH when n and m are equal to I or 2 and when Y represents a group CROH, CRNH₂, CRF or CF₂;
R₂ represents a radical —CO₂R, —CH₂CO₂R, —CH₂—CH₂OH, CH₂OH, CH₂—CH₂CO₂R, —CONH₂, —CH₂—CONH₂, —CH₂—CH₂—CONH₂, —CH₂—NH₂, —CH₂—CH₂—NH₂ or —CH₂—CH₂—CH₂—NH₂, R being as defined above;
R₃ represents a radical phenyl, heteroaryl, alk-R^{o}₃ for which alk is an alkylene radical and
R^{o}₃ represents hydrogen, halogen, hydroxyl, alkyloxy, alkylthio, alkylsulfinyl, alkylsulfonyl, alkylamino, dialkylamino, cycloalkyl, cycloalkyloxy, cycloalkylthio, cycloalkylsulfinyl, cycloalkylsulfonyl, cycloalkylamino, N-cycloalkyl-N-alkylamino, —N-(cyclo-alkyl)₂, acyl, cycloalkylcarbonyl, phenyl, phenoxy, phenylthio, phenylsulfinyl, phenylsulfonyl, phenyl-amino, N-alkyl-N-phenylamino, N-cycloalkyl-N-phenyl-amino, —N-(phenyl)₂, phenylalkyloxy, phenylalkylthio, phenylalkylsulfinyl, phenylalkylsulfonyl, phenylalkyl-amino, N-alkyl-N-phenylaminoalkyl, N-cycloalkyl-N-phenylalkylamino, benzoyl, heteroaryl, heteroaryloxy, heteroarylthio, heteroarylsulfinyl, heteroaryl-sulfonyl, heteroarylamino, N-alkyl-N-heteroarylamino, N-cycloalkyl-N-heteroarylamino, heteroarylcarbonyl, heteroarylalkyloxy, heteroarylalkylthio, heteroaryl-alkylsulfinyl, heteroarylalkylsulfonyl, heteroaryl-alkylamino, N-alkyl-N-heteroarylaminoalkyl, N-cyclo-alkyl-N-heteroarylaminoalkyl (the heteroaryl parts mentioned above being mono- or bicyclic), carboxyl, alkyloxycarbonyl, —NRaRb or —CO—NRaRb for which Ra and Rb respectively represent hydrogen, alkyl, cycloalkyl, phenyl, mono- or bicyclic heteroaryl, or one of Ra or Rb represents hydroxyl, alkyloxy, cycloalkyloxy, or Ra and Rb form together with the nitrogen atom to which they are attached a 5- or 6-membered heterocycle which may optionally contain another heteroatom chosen from O, S and N and carrying, where appropriate, an alkyl, phenyl or mono- or bicyclic heteroaryl substituent on the nitrogen atom or where appropriate in which the sulfur atom is oxidized to the sulfinyl or sulfonyl state,
or alternatively R^{o}₃ represents —CR′b=CR+c-R+a for which R′a represents phenyl, phenylalkyl, heteroaryl, heteroarylalkyl, phenoxyalkyl, phenylthioalkyl, phenylsulfinylalkyl, phenylsulfonylalkyl, phenylaminoalkyl, N-alkyl-N-phenylaminoalkyl, heteroaryloxyalkyl, hetero-arylthioalkyl, heteroarylsulfinylalkyl, heteroarylsulfonylalkyl, heteroarylaminoalkyl, N-alkyl-N-heteroarylaminoalkyl, heteroarylthio, heteroarylsulfinyl, heteroarylsulfonyl, (the heteroaryl parts mentioned above being mono- or bicyclic), phenylthio, phenylsulfinyl, phenylsulfonyl, and for which R′b and R′c represent hydrogen, alkyl or cycloalkyl,
or alternatively R₃ represents a radical —C≡C—Rd for which Rd is alkyl, phenyl, phenylalkyl, phenoxyalkyl, phenylthioalkyl, N-alkyl-N-phenylaminoalkyl, hetero-aryl, heteroarylalkyl, heteroaryloxyalkyl, hetero-arylthioalkyl, heteroarylaminoalkyl, N-alkyl-N-heteroarylaminoalkyl, (the heteroaryl parts mentioned above being mono- or bicyclic),
or alternatively R^{o}₃ represents a radical —CF₂-phenyl or mono- or bicyclic —CF₂-heteroaryl, it being understood that the phenyl, benzyl, benzoyl or heteroaryl radicals or portions mentioned above are optionally substituted on the ring with 1 to 4 substituents chosen from halogen, hydroxyl, alkyl, alkyloxy, alkyloxyalkyl, haloalkyl, trifluoromethyl, trifluoromethoxy, trifluoromethylthio, carboxyl, alkyloxycarbonyl, cyano, alkylamino, —NRaRb for which Ra and Rb are as defined above, phenyl, hydroxyalkyl, alkylthioalkyl, alkylsulfinylalkyl, alkylsulfonylalkyl;
R₄ represents a hydrogen atom or an alkyl radical optionally substituted with R₆, where R₆ represents an OH, NH2 or COOH radical, or a fluorine atom; and
R₅ is a hydrogen atom or an alkyl group;
it being understood that the alkyl or acyl radicals and portions contain (unless specifically stated) 1 to 10 carbon atoms in the form of a straight or branched chain and that the cycloalkyl radicals contain 3 to 6 carbon atoms;
in its enantiomeric or diastereoisomeric forms or mixtures of these forms, and/or where appropriate in E or Z form or mixtures thereof, and its salts.

2. The derivative of general formula (I) as defined in claim 1, wherein:
R₁, R′₁, R′₂, R′₃, R′₄ and R′₅ are identical or different and represent a hydrogen or halogen atom or an alkyl or alkyloxy radical, or represent a methylene radical substituted with alkyloxy;
m and n are equal to 1 or 2; and
R₃ represents a radical alk-R^{o}₃ for which alk is an alkylene radical and R^{o}₃ represents alkyloxy, alkylthio, alkylamino, dialkylamino, cycloalkyloxy, cycloalkylthio, cycloalkylamino, N-cycloalkyl-N-alkylamino, —N-(cycloalkyl)₂, phenyl, phenoxy, phenylthio, phenylamino, N-alkyl-N-phenylamino, N-cycloalkyl-N-phenylamino, phenylalkyloxy, phenyl-alkylthio, phenyl-alkylamino, N-alkyl-N-phenyl-aminoalkyl, N-cycloalkyl-N-phenylalkylamino, hetero-aryloxy, heteroarylthio, heteroarylamino, N-alkyl-N-heteroarylamino, N-cycloalkyl-N-heteroarylamino, heteroarylcarbonyl, heteroarylalkyloxy, heteroaryl-alkylthio, heteroarylalkylamino, N-alkyl-N-hetero-arylaminoalkyl, N-cycloalkyl-N-heteroarylaminoalkyl (the heteroaryl parts cited above being mono- or bicyclic), —NRaRb or —CO—NRaRb for which Ra and Rb are defined as in claim 1, or alternatively R^{o}₃ represents —CR′b=CR′c-R+a for which R′a represents phenyl, phenylalkyl, heteroaryl or heteroarylalkyl, phenoxyalkyl, phenylthioalkyl, phenylaminoalkyl, N-alkyl-N-phenylaminoalkyl, heteroaryloxyalkyl, heteroarylthioalkyl, heteroarylaminoalkyl, N-alkyl-N-heteroarylaminoalkyl, heteroarylthio, (the heteroaryl parts cited above being mono- or bicyclic), or phenylthio, and for which R′b and R′c represent hydrogen, alkyl or cycloalkyl, or alternatively R^{o}₃ represents a radical —C≡C—Rd for which Rd is alkyl, phenyl, phenylalkyl, phenoxyalkyl, phenylthioalkyl, N-alkyl-N-phenylaminoalkyl, heteroaryl, heteroarylalkyl, heteroaryloxyalkyl, heteroarylthioalkyl, heteroarylaminoalkyl, N-alkyl-N-heteroarylaminoalkyl, (the heteroaryl parts cited above being mono- or bicyclic),
or alternatively R^{o}₃ represents a radical —CF₂-phenyl or mono- or bicyclic —CF₂-heteroaryl;
it being understood that the phenyl, benzyl, benzoyl or heteroaryl radicals or portions mentioned above may be optionally substituted as envisaged in claim 1;
R₂, R₄, R₅, Y and Z are as defined in claim 1;
in its enantiomeric or diastereoisomeric forms or mixtures of these forms, and/or where appropriate in E or Z form or mixtures thereof, and its salts.

3. The derivative of general formula (I) as defined in claim 1, wherein:
R₁, R′₁, R′₂, R′₃, R′₄ and R′₅ are identical or different and represent a hydrogen or halogen atom or an alkyl or alkyloxy radical, or represent a methylene radical substituted with alkyloxy;
m and n are equal to 1;
Y represents a group CH₂, CHOH, CHF, CHNH₂ or C=O;
R₂ represents a radical COOR, CH₂—COOR, CH₂OH or CH₂CH₂OH, R being as defined in claim 1;
Z represents a group CH₂;
R₃ represents a radical alk-R^{o}₃ for which alk is an alkylene radical and R^{o}₃ represents cycloalkyloxy, cycloalkylthio, phenyl, phenoxy, phenylthio, phenylalkyloxy, phenylalkylthio, heteroaryloxy, heteroarylthio, heteroarylalkyloxy, heteroaryl-alkylthio, (the heteroaryl parts cited above being mono- or bicyclic),
or alternatively R^{o}₃ represents —CR′b=CR′c-R′a for which R′a represents phenyl, phenylthioalkyl, heteroaryl, heteroarylalkyl, phenoxyalkyl, phenyl-thioalkyl, heteroaryloxyalkyl, heteroarylthioalkyl (the heteroaryl parts cited above being mono- or bicyclic), or phenylthio, and for which R′b and R′c represent hydrogen, alkyl or cycloalkyl,
or alternatively R^{o}₃ represents a radical —C≡C—Rd for which Rd is alkyl, phenyl, phenylalkyl, phenoxyalkyl, phenylthioalkyl, N-alkyl-N-phenylaminoalkyl, mono- or bicyclic heteroaryl, heteroarylalkyl, heteroaryloxyalkyl, heteroarylthioalkyl, (the heteroaryl parts cited above being mono- or bicyclic);
R₄ represents a hydrogen atom or an alkyl radical optionally substituted with R₆, where R₆ represents an OH radical or a fluorine atom;
R₅ is a hydrogen atom or an alkyl group;
it being understood that the phenyl, benzyl, benzoyl or heteroaryl radicals or portions mentioned above may be optionally substituted as envisaged above;
in its enantiomeric or diastereoisomeric forms or mixtures of these forms, and/or where appropriate in Z or E form or mixtures thereof, and its salts.

4. The derivative of general formula (I) as defined in claim 3, wherein:
Y and Z represent a group CH₂;
R₂ represents a radical COOR or CH₂—COOR, R being as defined in claim 1;
R₅ is a hydrogen atom;

5. Any one of the derivatives of general formula (I) as claimed in claim 1, whose names follow:
ethyl (RS)-2-{[(E)-3-(2,5-difluorophenyl)allylamino]methyl}-5-(3-fluoro-6-methoxyquinolin-4-yl)pentanoate;
ethyl (RS)-2-{[(E)-3-(2,5-difluorophenyl)allylamino]methyl}-5-(3-fluoro-6-methoxyquinolin-4-yl)pentanoate;
(RS)-2-{[(E)-3-(2,5-difluorophenyl)allylamino]methyl}-5-(3 -fluoro-6-methoxyquinolin-4-yl)pentanoic acid;
2-{[(E)-3 -(2,5-difluorophenyl)allylamino]methyl}-5-(3-fluoro-6-methoxyquinolin-4-yl)pentanoic acid;
2-{[(E)-3-(2,5-difluorophenyl)allylamino]methyl }-5-(3-fluoro-6-methoxyquinolin-4-yl)pentanoic acid;
(RS)-2-{[3 -(2,5-difluorophenyl)propylamino]methyl}-5-(3-fluoro-6-methoxyquinolin-4-yl)pentanoic acid;
ethyl (RS)-2-({N-[(E)-3-(2,5-difluorophenyl)allyl]-N-methylamino}methyl)-5-(3-fluoro-6-methoxyquinolin-4-yl)pentanoate;
sodium (RS)-2-({N-[(E)-3-(2,5-difluorophenyl)allyl]-N-methylamino}methyl)-5-(3-fluoro-6-methoxyquinolin-4-yl)pentanoate;
(RS)-5-(3-fluoro-6-methoxyquinolin-4-yl)-2-{[2-(thiophen-2-ylsulfanyl)ethylamino]methyl}pentanoic acid;
(RS)-2-{[2-(2,5-difluorophenylsulfanyl)ethylamino]methyl}-5-(3-fluoro-6-methoxyquinolin-4-yl)pentanoic acid;
(RS)-2-{[2-(2,5-difluorophenoxy)ethylamino]methyl}-5-(3-fluoro-6-methoxyquinolin-4-yl)pentanoic acid;
(RS)-2-{[N-[(E)-3-(2,5-difluorophenyl)allyl]-N-(2-fluoroethyl)amino]methyl}-5-(3-fluoro-6-methoxyquinolin-4-yl)pentanoic acid;
(RS)-2-{[N-[(E)-3-(2,5-difluorophenyl)allyl]-N-(2-hydroxyethyl)amino]methyl}-5-(3-fluoro-6-methoxyquinolin-4-yl)pentanoic acid;
in its enantiomeric or diastereoisomeric forms or mixtures of these forms, and/or where appropriate in Z or E form or mixtures thereof, and its salts.

6. A method for preparing the derivatives of general formula (I) as defined in claim 1, wherein the chain R₃ defined in claim 1 is condensed with the 4-substituted quinoline derivative of general formula (II) 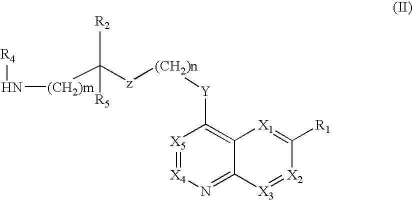
in which X₁, X₂, X₃, X₄, X₅, R₁, R₂, Y, Z, m, n, R₄ and R₅ are as defined in claim 1, R₂ being protected when it carries a carboxyl radical, and then where appropriate the group protecting the carboxyl radical is removed, optionally the enantiomeric and diastereoisomeric forms and/or where appropriate the Z or E forms are separated and optionally the product obtained is converted to a salt.

7. The method as claimed in claim 6, wherein the condensation of the chain R₃ with the nitrogen is carried out by the action of a derivative of general formula (IIa):
R₃-X (IIa)
in which R₃ is defined as in claim 1 and X represents a halogen atom, a methylsulfonyl radical, a trifluoromethylsulfonyl radical or a p-toluenesulfonyl radical.

8. The method as claimed in claim 6, wherein when R₃ represents a radical -alk-R^{o}₃ for which alk is an alkyl radical and R^{o}₃ represents a radical —C≡C—Rd in which Rd is as defined in claim 1, a condensation of an alkynyl halide HC≡C-alk-X for which alk is defined as above and X is a halogen atom is carried out, followed by substitution of the chain with an appropriate radical Rd.

9. The method as claimed in claim 6, wherein when R₃ represents a radical -alk-R^{o}₃ for which alk is an alkyl radical and R^{o}₃ represents a phenoxy, phenylthio, phenylamino, heteroaryloxy, heteroarylthio or heteroarylamino radical, the reaction is carried out by constructing the chain by first condensing a chain HO-alk-X for which X is a halogen atom, and then either by converting the hydroxyalkyl chain obtained to a haloalkyl, methanesulfonylalkyl or p-toluenesulfonylalkyl chain and finally by causing an aromatic derivative having the structure R₃H or R₃H₂ to act in a basic medium, or by causing the aromatic derivative to act directly under dehydration conditions.

10. The method as claimed in claim 6 for the preparation of compounds of general formula (I) in which R₄ represents an alkyl group optionally substituted with R₆, a product of general formula (I) where R₄ represents a hydrogen atom being subjected to the action of appropriate alkylating reagents.

11. The method as claimed in claim 6, wherein the derivatives of general formula (II) for which Y is a group CHR, Z is a group CH₂ and m and n are defined as in the preceding claims, are prepared by condensing a heteroaromatic derivative of general formula (III): 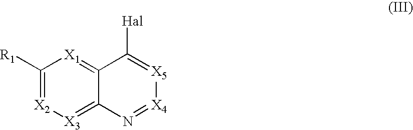
in which R₁, X₁, X₂, X₃, X₄ and X₅ are defined as in claim 1 and Hal represents a halogen atom, with a derivative of general formula (IV): 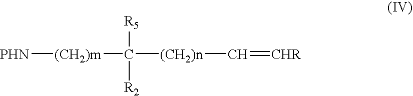
in which P is a group protecting the amino functional group and R, m, n, R₅ and R₂ are defined as in claim 1 or R₂ represents a protected radical if R₂ represents or carries a carboxylic acid functional group, followed by the removal of the protecting groups and/or followed by the conversion, by a subsequent operation, of the substituents of the aromatic bicycle of general formula (II) thus obtained, to give the expected derivative carrying the radical R₁, R′₁, R′₂, R′₃, R′₄, R′₅, and where appropriate removing the protecting radical(s) still present in the molecule.

12. The derivatives of general formula (II) as defined in claim 6.

13. The derivatives of general formula (IV) as defined in claim 11.

14. As medicaments, the derivatives of general formula (I) as defined in claim 1.

15. As medicaments, the derivatives of general formula (I) as defined in claim 2.

16. A pharmaceutical composition, which contains at least one medicament as claimed in claim 1, in the pure state or in combination with one or more compatible and pharmaceutically acceptable diluents and/or adjuvants.
